(19) United States
(12) Patent Application Publication
(10) Pub. No.: US 2025/0002656 A1
(43) Pub. Date: Jan. 2, 2025

(54) COMPOSITION FOR A MOLDED BODY

(71) Applicant: BOLT THREADS, INC., Emeryville, CA (US)

(72) Inventors: Lindsay Wray, Benicia, CA (US); Nour Eldien El-Difrawy, Berkeley, CA (US); Paul Andre Guerette, Berkeley, CA (US); Maxime Boulet-Audet, El Cerrito, CA (US); Gregory Wilson Rice, South San Francisco, CA (US); Joshua Tyler Kittleson, Pleasant Hill, CA (US); Jeroen Visjager, Cincinnati, OH (US)

(21) Appl. No.: 18/825,861

(22) Filed: Sep. 5, 2024

Related U.S. Application Data
(63) Continuation of application No. 16/538,519, filed on Aug. 12, 2019, now abandoned.
(60) Provisional application No. 62/717,622, filed on Aug. 10, 2018.

(51) Int. Cl. C08H 1/00; C08K 5/053; D01D 5/08

(52) U.S. Cl. CPC C08H 1/00; C08K 5/053; D01D 5/08; D10B 2211/04

(57) ABSTRACT

The present disclosure relates to a composition for a molded body comprising a recombinant spider silk protein, and a plasticizer. Further, the present disclosure relates to a molded body comprising a recombinant spider silk protein and a plasticizer, and a process for preparing the molded body.

### CROSS-REFERENCE TO RELATED APPLICATIONS

This application is a continuation of U.S. patent application Ser. No. 16/538,519 filed Aug. 12, 2019, which claims the benefit of U.S. Provisional Application No. 62/717,622, filed Aug. 10, 2018, the entire contents of which are incorporated by herein by reference for all purposes.

### SEQUENCE LISTING

A Sequence Listing, which is a part of the present disclosure, is submitted concurrently with the specification as a text file. The name of the text file containing the Sequence Listing is “50017A_SeqListing.xml”. The Sequence Listing was created on Sep. 5, 2024, and is 152,360 bytes in size. The subject matter of the Sequence Listing is incorporated herein in its entirety by reference.

### FIELD

The present disclosure relates to a composition for a molded body comprising a recombinant spider silk protein, and a plasticizer. Further, the present disclosure relates to a molded body comprising a recombinant spider silk protein and a plasticizer, and a process for preparing the molded body.

### BACKGROUND

Biorenewable and biodegradable materials are of increasing interest as an alternative to petroleum-based products. To this end, considerable effort has been made to develop methods of making materials and fibers from molecules derived from plants and animals. Fiber made from regenerated protein dates back to the 1890s and has been made using various traditional wet-spinning techniques.

Wet spinning uses both solvents and coagulation baths to produce fiber. This is disadvantageous in that the chemicals used as solvents and in coagulation baths need to be extracted from the fiber after the spinning process and subject to a closed loop process in order to provide a sustainable and responsible process. While melt spinning provides an attractive option to wet spinning in that solvent and coagulation baths are not required, melt spinning also requires that (i) the polymer should produce a homogeneous melt composition that can be extruded to form a commercial-quality fiber, and (ii) the polymer should not be degraded during the melting and extrusion steps.

### SUMMARY

Provided herein, according to some embodiments of the invention, are a composition for a molded body, and a molded body, comprising a recombinant spider silk protein, and a plasticizer, wherein the composition may be substantially homogeneous after being transformed into a melted or flowable state; and the recombinant spider silk protein is substantially non-degraded, or degraded in an amount of less than 6.0 weight % after it is formed into a molded body.

Further, the present disclosure provides a process for preparing a molded body, comprising the steps of applying pressure and/or shear force to a composition comprising a recombinant spider silk protein and a plasticizer to form a substantially homogeneous melt composition, and molding the homogeneous melt composition to form the molded body. The substantially homogeneous melt composition will typically be in a flowable state and may be extruded, for instance to form fibers.

According to some embodiments, provided herein is a composition for a molded body comprising a recombinant spider silk protein and a plasticizer, wherein the composition is capable of being induced into a flowable state, wherein the recombinant spider silk protein is substantially non-degraded in the flowable state.

In some embodiments, the composition is capable of being induced into the flowable state by the application of shear force and pressure. In some embodiments, the composition is capable of being induced into the flowable state by the application of shear force and pressure without the application of heat. In some embodiments, the composition is capable of being induced into the flowable state and extruded multiple times with the recombinant spider silk protein remaining substantially non-degraded within the composition.

In some embodiments, the composition is thermoplastic.

In some embodiments, the composition is capable of being induced into the flowable state through the application of shear force ranging from 1.5 Nm to 13 Nm. In some embodiments, the composition is capable of being induced into the flowable state through the application of shear force ranging from 2 Nm to 6 Nm. In some embodiments, the composition is capable of being induced into the flowable state through the application of pressure ranging from 1 MPa to 300 MPa. In some embodiments, the composition is capable of being induced into the flowable state through the application of pressure ranging from 5 MPa to 75 MPa.

In some embodiments, the composition is capable of being induced into the flowable state at less than 120° C., less than 80° C., less than 40° C., or at room temperature. In some embodiments, the composition is substantially homogeneous.

In some embodiments, the recombinant spider silk protein comprises repeat units. In some embodiments, the recombinant spider silk protein comprises in the range 2 to 20 repeat units of amino acid residue length ranging from 60 to 100 amino acids. In some embodiments, the molecular weight of the recombinant spider silk protein ranges from 20 to 2000 kDa.

In some embodiments, the recombinant spider silk protein comprises at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; and a glycine-rich region with 12 or more consecutive amino acids, comprising a glycine content of at least 40% and an alanine content of less than 30%.

In some embodiments, the plasticizer is selected from a polyol, water and/or urea. In some embodiments, the polyol comprises glycerol. In some embodiments, the plasticizer comprises water. In some embodiments, the recombinant spider silk protein is present in a recombinant spider silk polypeptide powder and wherein the ratio by weight of plasticizer to recombinant silk polypeptide powder ranges from 0.05 to 1.50:1. In some embodiments, the recombinant spider silk protein is present in a recombinant spider silk polypeptide powder and the ratio by weight of plasticizer to recombinant silk polypeptide powder ranges from 0.20 to 0.70:1.

In some embodiments, the recombinant spider silk protein is present in a recombinant spider silk polypeptide powder and the amount of recombinant spider silk polypeptide powder in the composition ranges from 1 to 90 wt % recombinant spider silk protein. In some embodiments, the recombinant spider silk protein is present in a recombinant spider silk polypeptide powder and the amount of recombinant spider silk polypeptide powder in the composition ranges from 20 to 41 wt % recombinant spider silk protein. In some embodiments, the composition comprises in the range 1 to 60 wt % of glycerol as a plasticizer. In some embodiments, the composition comprises in the range 15 to 30wt % of glycerol as a plasticizer. In some embodiments, the composition comprises in the range 5 to 80 wt % of water as a plasticizer. In some embodiments, the composition comprises in the range 19 to 27 wt % of water as a plasticizer.

In some embodiments, the recombinant spider silk protein is degraded in an amount of less than 10.0 weight % in the flowable state. In some embodiments, the recombinant spider silk protein is degraded in an amount of less than 6.0 weight % in the flowable state. In some embodiments, the recombinant spider silk protein is degraded in an amount of less than 2.0 weight % in flowable state. In some embodiments, the degradation of the recombinant spider silk protein is assessed by measuring the amount of full-length recombinant spider silk protein present in the composition before and after the flowable state is induced. In some embodiments, the amount of full-length recombinant spider silk protein is measured using size exclusion chromatography.

Also provided herein, according to some embodiments of the invention, is a molded body comprising the composition for a molded body comprising a recombinant spider silk protein and a plasticizer, wherein the composition is capable of being induced into a flowable state, wherein the recombinant spider silk protein is substantially non-degraded in the flowable state.

In some embodiments, the molded body is a fiber. In some embodiments, the fiber has a strength in the range of 100 Pa to 1.2 GPa. In some embodiments, the fiber is of birefringence in the range from 5×10−5 to ˜0.04 as measured by polarized light microscopy.

Also provided herein, according to some embodiments of the invention, is a process for preparing a molded body, comprising the steps of: applying pressure and shear force to a composition comprising a recombinant spider silk protein and a plasticizer to transform the composition to a flowable state, and extruding the composition in the flowable state to form a molded body.

In some embodiments, extruding the composition to form a molded body comprises extruding the composition to form a fiber. In some embodiments, extruding the composition to form a fiber comprises extruding the composition through a spinneret. In some embodiments, extruding the composition to form a molded body comprises extruding the composition into a mold.

In some embodiments, the process for preparing a molded body further comprises: (a) applying pressure and shear force to the molded body to transform the molded body to a composition in a flowable state, and (b) extruding the composition in the flowable state to form a second molded body. In some embodiments, the process further comprises repeating steps (a) and (b) to the second molded body at least once.

In some embodiments, the shear force is from 1.5 to 13 N*m. In some embodiments, the pressure is from 1 MPa to 300 MPa. In some embodiments, the shear force and pressure are applied to the composition using a capillary rheometer or a twin screw extruder. In some embodiments, the screw speed of the twin screw extruder ranges from 10 to 300 RPM during application of said pressure and shear force.

In some embodiments, an instrument used to apply the shear force and pressure comprises a mixing chamber that is coupled to and proximal to an extrusion chamber. In some embodiments, the composition is heated in the mixing chamber. In some embodiments, the composition is heated in the extrusion chamber. In some embodiments, the composition is heated to a temperature of less than 120° C. In some embodiments, the composition is heated to a temperature of less than 80° C. In some embodiments, the composition is heated to a temperature of less than 40° C. In some embodiments, the extrusion chamber is tapered proximal to an orifice through which the composition is extruded. In some embodiments, the extrusion chamber is temperature controlled. In some embodiments, the composition has a residence time in the mixing chamber ranging from 3 to 7 minutes.

In some embodiments, the molded body after extrusion has a loss of water content of less than 15% as compared to the composition before extrusion. In some embodiments, the molded body after extrusion has a loss of water content of less than 10% as compared to the composition before extrusion.

In some embodiments, the molded body is a fiber and the fiber is hand drawn. In some embodiments, the molded body is a fiber and the fiber is drawn over multiple steps.

In some embodiments, the recombinant spider silk protein is substantially non-degraded in the molded body. In some embodiments, the recombinant spider silk protein is degraded in amount of less than 10% by weight in the molded body. In some embodiments, the recombinant spider silk protein is degraded in amount of less than 6% by weight in the molded body. In some embodiments, the recombinant spider silk protein is degraded in amount of less than 2% by weight in the molded body. In some embodiments, the degradation of the recombinant spider silk protein is assessed by measuring the amount of full-length recombinant spider silk protein present in the composition before and after extrusion. In some embodiments, the amount of full-length recombinant spider silk protein is measured using size exclusion chromatography.

In some embodiments, the molded body has minimal birefringence as measured by polarized light microscopy.

### BRIEF DESCRIPTION OF THE DRAWINGS

The foregoing and other objects, features and advantages will be apparent from the following description of particular embodiments of the invention, as illustrated in the accompanying drawings.

FIG. 1 shows Size Exclusion Chromatography data for P49W21G30 melt compositions extruded under selected heat and RPM conditions, according to various embodiments of the present invention.

FIG. 2 shows Size Exclusion Chromatography data for P65W20G15 melt compositions extruded under selected heat and RPM conditions, according to various embodiments of the present invention

FIG. 3 shows Size Exclusion Chromatography data for P71W19G10 melt compositions extruded under selected heat and RPM conditions, according to various embodiments of the present invention.

FIG. 4 shows a chart of water loss during extrusion for P49W21G30 melt compositions extruded under selected heat and RPM conditions as measured by thermogravimetric analysis (TGA), according to various embodiments of the invention. The data shows % water content of the starting pellet before extrusion and in samples extruded under selected conditions after extrusion.

FIG. 5 shows a chart of water loss during extrusion for P65W20G15 melt compositions extruded under selected heat and RPM conditions as measured by thermogravimetric analysis (TGA), according to various embodiments of the invention. The data shows % water content of the starting pellet before extrusion and in samples extruded under selected conditions after extrusion.

FIG. 6 shows a chart of water loss during extrusion for P71W19G10 melt compositions extruded under selected heat and RPM conditions as measured by thermogravimetric analysis (TGA), according to various embodiments of the invention. The data shows % water content of the starting powder before extrusion and in samples extruded under selected conditions after extrusion.

FIG. 7 shows beta sheet content for P49W21G30 samples extruded under selected heat and RPM conditions as measured by Fourier Transform Infrared Spectroscopy (FTIR). The samples were compared to reference controls of starting protein powder and starting pellets.

FIG. 8 shows beta sheet content for P65W20G15 samples extruded under selected heat and RPM conditions as measured by Fourier Transform Infrared Spectroscopy (FTIR). The samples were compared to reference controls of starting protein powder and starting pellets.

FIG. 9 shows beta sheet content for P71W19G10 samples extruded under selected heat and RPM conditions as measured by Fourier Transform Infrared Spectroscopy (FTIR). The samples were compared to reference controls of starting protein powder and starting pellets.

FIG. 10 shows images of selected extrusion products produced at 20° C. at 10, 100, 200 or 300 RPM captured using polarized light microscopy.

FIG. 11 shows images of selected extrusion products produced at 95° C. at 10, 100, 200 or 300 RPM captured using polarized light microscopy.

FIG. 12 shows a chart of glycerol loss during extrusion for P49W21G30 extrudates extruded under selected heat and RPM conditions as measured by HPLC, according to various embodiments of the invention. The data shows % glycerol content of the starting powder or pellet before extrusion and in samples after extrusion under selected conditions.

FIG. 13 shows a chart of glycerol loss during extrusion for P65W20G15 extrudates extruded under selected heat and RPM conditions as measured by HPLC, according to various embodiments of the invention. The data shows % glycerol content of the starting powder or pellet before extrusion and in samples after extrusion under selected conditions.

FIG. 14 shows a chart of glycerol loss during extrusion for P71W19G10 extrudates extruded under selected heat and RPM conditions as measured by HPLC, according to various embodiments of the invention. The data shows % glycerol content of the starting powder or pellet before extrusion and in samples after extrusion under selected conditions.

### DETAILED DESCRIPTION

The details of various embodiments of the invention are set forth in the description below. Other features, objects, and advantages of the invention will be apparent from the description. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

### Definitions

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

The term “molded body” as defined herein refers to a body manufactured by shaping liquid or pliable raw material using a rigid frame called a mold, such as the molding process including but not limited to extrusion molding, injection molding, compression molding, blow molding, laminating, matrix molding, rotational molding, spin casting, transfer molding, thermoforming, and the like.

The term “fiber” as defined herein refers to a molded body that is elongate, typically a fiber will have the form of a filament.

The term “melt spinning” as used herein refers to a method of forming fibers from a polymer wherein the polymer is transformed into a meltable or flowable state, and then solidifies by cooling after being extruded from the spinneret.

The term “drawing” as used herein with reference to a fiber refers to the application of force to stretch a spun fiber along its longitudinal axis during or after extrusion of the fiber. The term “undrawn fibers” refers to fibers that have been extruded but have not been subject to any drawing. The term “draw ratio” is a term of art commonly defined as the ratio between the collection rate and the feeding rate. At constant volume, it can be determined from a ratio of the initial diameter (Dᵢ) and final diameter (D_{f}) of the fiber (i.e., Dᵢ/D_{f}).

The term “glass transition” as used herein refers to the transition of a substance or composition from a hard, rigid or “glassy” state into a more pliable, “rubbery” or “viscous” state.

The term “glass transition temperature” as used herein refers to the temperature at which a substance or composition undergoes a glass transition.

The term “melt transition” as used herein refers to the transition of a substance or composition from a rubbery state to a less-ordered liquid phase or flowable state.

The term “melting temperature” as used herein refers to the temperature range over which a substance undergoes a melt transition.

The term “plasticizer” as used herein refers to any molecule that interacts with a polypeptide sequence to prevent the polypeptide sequence from forming tertiary structures and bonds and/or increases the mobility of the polypeptide sequence.

The term “flowable state” as used herein refers to a composition that has characteristics that are substantially the same as liquid (i.e. has transitioned from a rubbery state into a more liquid state).

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Overview

Provided herein is a composition for a molded body, comprising a recombinant spider silk protein, and a plasticizer, wherein the composition is homogeneous or substantially homogeneous in a melted or flowable state; and the recombinant spider silk protein is substantially non-degraded after it is formed into a molded body (e.g. degraded in an amount of less than 10%, or often less than 6% by weight).

### Recombinant Silk Proteins

The present disclosure describes embodiments of the invention including fibers synthesized from synthetic proteinaceous copolymers (i.e., recombinant polypeptides). Suitable proteinaceous co-polymers are discussed in U.S. Patent Publication No. 2016/0222174, published Aug. 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides capable of molded body formation.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block co-polymer polypeptides having sequence similarity and/or amino acid composition similarity to the repeat domains of native silk proteins can be used to manufacture on commercial scales consistent molded bodies that have properties that recapitulate the properties of corresponding molded bodies made from natural silk polypeptides.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of molded body formation.

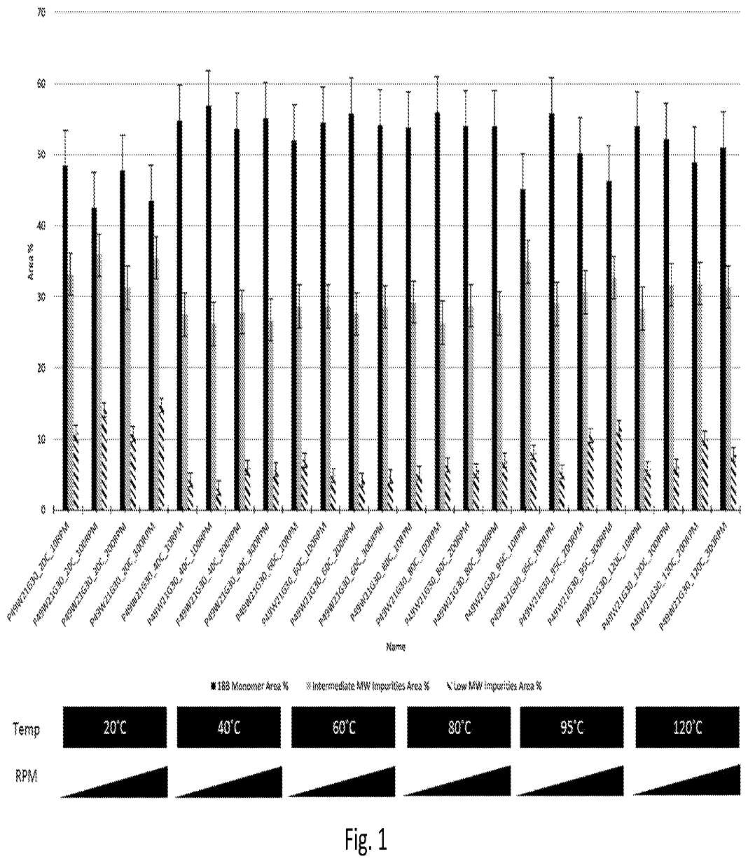

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture, as depicted in FIG. 1. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1A lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 3) is, for the purposes of the present invention, the same as GSGAG (SEQ ID NO: 4) and the same as GGSGA (SEQ ID NO: 5); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1A
Samples of Block Sequences

| Species | Silk Type | Representative Block Amino Acid Sequence |
|---|---|---|
| Aliatypus gulosus | Fibroin 1 | GAASSSSTIITTKSASASAAADASAAATASAASRSSANAAASAFAQS |
| | | FSSILLESGYFCSIFGSSISSSYAAAIASAASRAAAESNGYTTHAYA |
| | | CAKAVASAVERVTSGADAYAYAQAISDALSHALLYTGRLNTANANSL |
| | | ASAFAYAFANAAAQASASSASAGAASASGAASASGAGSAS |
| | | (SEQ ID NO: 6) |
| Plectreurys tristis | Fibroin 1 | GAGAGAGAGAGAGAGAGSGASTSVSTSSSSGSGAGAGAGSGAGSGAG |
| | | AGSGAGAGAGAGGAGAGFGSGLGLGYGVGLSSAQAQAQAQAAAQAQA |
| | | QAQAQAYAAAQAQAQAQAQAQAAAAAAAAAAA |
| | | (SEQ ID NO: 7) |
| Plectreurys tristis | Fibroin 4 | GAAQKQPSGESSVATASAAATSVTSGGAPVGKPGVPAPIFYPQGPLQ |
| | | QGPAPGPSNVQPGTSQQGPIGGVGGSNAFSSSFASALSLNRGFTEVI |
| | | SSASATAVASAFQKGLAPYGTAFALSAASAAADAYNSIGSGANAFAY |
| | | AQAFARVLYPLVQQYGLSSSAKASAFASAIASSFSSGTSGQGPSIGQ |
| | | QQPPVTISAASASAGASAAAVGGGQVGQGPYGGQQQSTAASASAAAA |
| | | TATS (SEQ ID NO: 8) |
| Araneus | TuSp | GNVGYQLGLKVANSLGLGNAQALASSLSQAVSAVGVGASSNAYANAV |
| gemmoides | | SNAVGQVLAGQGILNAANAGSLASSFASALSSSAASVASQSASQSQA |
| | | ASQSQAAASAFRQAASQSASQSDSRAGSQSSTKTTSTSTSGSQADSR |
| | | SASSSASQASASAFAQQSSASLSSSSSFSSAFSSATSISAV |
| | | (SEQ ID NO: 9) |
| Argiope aurantia | TuSp | GSLASSFASALSASAASVASSAAAQAASQSQAAASAFSRAASQSASQ |
| | | SAARSGAQSISTTTTTSTAGSQAASQSASSAASQASASSFARASSAS |
| | | LAASSSFSSAFSSANSLSALGNVGYQLGFNVANNLGIGNAAGLGNAL |
| | | SQAVSSVGVGASSSTYANAVSNAVGQFLAGQGILNAANA |
| | | (SEQ ID NO: 10) |
| Deinopis spinosa | TuSp | GASASAYASAISNAVGPYLYGLGLFNQANAASFASSFASAVSSAVAS |
| | | ASASAASSAYAQSAAAQAQAASSAFSQAAAQSAAAASAGASAGAGAS |
| | | AGAGAVAGAGAVAGAGAVAGASAAAASQAAASSSASAVASAFAQSAS |
| | | YALASSSAFANAFASATSAGYLGSLAYQLGLTTAYNLGLSNAQAFAS |
| | | TLSQAVTGVGL (SEQ ID NO: 11) |
| Nephila clavipes | TuSp | GATAASYGNALSTAAAQFFATAGLLNAGNASALASSFARAFSASAES |
| | | QSFAQSQAFQQASAFQQAASRSASQSAAEAGSTSSSTTTTTSAARSQ |
| | | AASQSASSSYSSAFAQAASSSLATSSALSRAFSSVSSASAASSLAYS |
| | | IGLSAARSLGIADAAGLAGVLARAAGALGQ |
| | | (SEQ ID NO: 12) |
| Argiope trifasciata | Flag | GGAPGGGPGGAGPGGAGFGPGGGAGFGPGGGAGFGPGGAAGGPGGPG |
| | | GPGGPGGAGGYGPGGAGGYGPGGVGPGGAGGYGPGGAGGYGPGGSGP |
| | | GGAGPGGAGGEGPVTVDVDVTVGPEGVGGGPGGAGPGGAGFGPGGGA |
| | | GFGPGGAPGAPGGPGGPGGPGGPGGPGGVGPGGAGGYGPGGAGGVGP |
| | | AGTGGFGPGGAGGFGPGGAGGFGPGGAGGFGPAGAGGYGPGGVGPGG |
| | | AGGFGPGGVGPGGSGPGGAGGEGPVTVDVDVSV |
| | | (SEQ ID NO: 13) |
| Nephila clavipes | Flag | GVSYGPGGAGGPYGPGGPYGPGGEGPGGAGGPYGPGGVGPGGSGPGG |
| | | YGPGGAGPGGYGPGGSGPGGYGPGGSGPGGYGPGGSGPGGYGPGGSG |
| | | PGGYGPGGYGPGGSGPGGSGPGGSGPGGYGPGGTGPGGSGPGGYGPG |
| | | GSGPGGSGPGGYGPGGSGPGGFGPGGSGPGGYGPGGSGPGGAGPGOV |
| | | GPGGFGPGGAGPGGAAPGGAGPGGAGPGGAGPGGAGPGGAGPGGAGP |
| | | GGAGGAGGAGGSGGAGGSGGTTIIEDLDITIDGADGPITISEELPIS |
| | | GAGGSGPGGAGPGGVGPGGSGPGGVGPGGSGPGGVGPGGSGPGGVGP |
| | | GGAGGPYGPGGSGPGGAGGAGGPGGAYGPGGSYGPGGSGGPGGAGGP |
| | | YGPGGEGPGGAGGPYGPGGAGGPYGPGGAGGPYGPGGEGGPYGP |
| | | (SEQ ID NO: 14) |
| Latrodectus | AcSp | GINVDSDIGSVTSLILSGSTLQMTIPAGGDDLSGGYPGGFPAGAQPS |
| hesperus | | GGAPVDFGGPSAGGDVAAKLARSLASTLASSGVFRAAFNSRVSTPVA |
| | | VQLTDALVQKIASNLGLDYATASKLRKASQAVSKVRMGSDTNAYALA |
| | | ISSALAEVLSSSGKVADANINQIAPQLASGIVLGVSTTAPQFGVDLS |
| | | SINVNLDISNVARNMQASIQGGPAPITAEGPDFGAGYPGGAPTDLSG |
| | | LDMGAPSDGSRGGDATAKLLQALVPALLKSDVFRAIYKRGTRKQVVQ |
| | | YVTNSALQQAASSLGLDASTISQLQTKATQALSSVSADSDSTAYAKA |
| | | FGLAIAQVLGTSGQVNDANVNQIGAKLATGILRGSSAVAPRLGIDLS |
| | | (SEQ ID NO: 15) |
| Argiope trifasciata | AcSp | GAGYTGPSGPSTGPSGYPGPLGGGAPFGQSGFGGSAGPQGGFGATGG |
| | | ASAGLISRVANALANTSTLRTVLRTGVSQQIASSVVQRAAQSLASTL |
| | | GVDGNNLARFAVQAVSRLPAGSDTSAYAQAFSSALFNAGVLNASNID |
| | | TLGSRVLSALLNGVSSAAQGLGINVDSGSVQSDISSSSSFLSTSSSS |
| | | ASYSQASASSTS (SEQ ID NO: 16) |
| Uloborus diversus | AcSp | GASAADIATAIAASVATSLQSNGVLTASNVSQLSNQLASYVSSGLSS |
| | | TASSLGIQLGASLGAGFGASAGLSASTDISSSVEATSASTLSSSASS |
| | | TSVVSSINAQLVPALAQTAVLNAAFSNINTQNAIRIAELLTQQVGRQ |
| | | YGLSGSDVATASSQIRSALYSVQQGSASSAYVSAIVGPLITALSSRG |
| | | VVNASNSSQIASSLATAILQFTANVAPQFGISIPTSAVQSDLSTISQ |
| | | SLTAISSQTSSSVDSSTSAFGGISGPSGPSPYGPQPSGPTFGPGPSL |
| | | SGLTGFTATFASSFKSTLASSTQFQLIAQSNLDVQTRSSLISKVLIN |
| | | ALSSLGISASVASSIAASSSQSLLSVSA (SEQ ID NO: 17) |
| Euprosthenops | MaSp1 | GGQGGQGQGRYGQGAGSSAAAAAAAAAAAAAA |
| australis | | (SEQ ID NO: 18) |
| Tetragnatha | MaSp1 | GGLGGGQGAGQGGQQGAGQGGYGSGLGGAGQGASAAAAAAAA |
| kauaiensis | | (SEQ ID NO: 19) |
| Argiope aurantia | MaSp2 | GGYGPGAGQQGPGSQGPGSGGQQGPGGLGPYGPSAAAAAAAA |
| | | (SEQ ID NO: 20) |
| Deinopis spinosa | MaSp2 | GPGGYGGPGQQGPGQGQYGPGTGQQGQGPSGQQGPAGAAAAAAAAA |
| | | (SEQ ID NO: 21) |
| Nephila clavata | MaSp2 | GPGGYGLGQQGPGQQGPGQQGPAGYGPSGLSGPGGAAAAAAA |
| | | (SEQ ID NO: 22) |
| Deinopis Spinosa | MiSp | GAGYGAGAGAGGGAGAGTGYGGGAGYGTGSGAGYGAGVGYGAGAGAGG |
| | | GAGAGAGGGTGAGAGGGAGAGYGAGTGYGAGAGAGGGAGAGAGAGAGA |
| | | GAGAGSGAGAGYGAGAGYGAGAGAGGVAGAGAAGGAGAAGGAGAAGGA |
| | | GAAGGAGAGAGAGSGAGAGAGGGARAGAGG [SEQ ID NO: 23] |
| Latrodectus | MiSp | GGGYGRGQGAGAGVGAGAGAAAGAAAIARAGGYGQGAGGYGQGQGAGA |
| hesperus | | AAGAAAGAGAGGYGQGAGGYGRGQGAGAGAGAGAGARGYGQGAGAGAA |
| | | AGAAASAGAGGYGQGAGGYGQGQGAGAAAGAAASAGAGGYGQGAGGYG |
| | | QGQGA [SEQ ID NO: 24] |
| Nephila clavipes | MiSp | GAGAGGAGYGRGAGAGAGAAAGAGAGAAAGAGAGAGGYGGQGGYGAGA |
| | | GAGAAAAAGAGAGGAAGYSRGGRAGAAGAGAGAAAGAGAGAGGYGGQG |
| | | GYGAGAGAGAAAAAGAGSGGAGGYGRGAGAGAAAGAGAAAGAGAGAGG |
| | | YGGQGGYGAGAGAAAAA [SEQ ID NO: 25] |
| Nephilengys | MiSp | GAGAGVGGAGGYGSGAGAGAGAGAGAASGAAAGAAAGAGAGGAGGYGT |
| cruentata | | GQGYGAGAGAGAGAGAGGAGGYGRGAGAGAGAGAGGAGGYGAGQGYGA |
| | | GAGAGAAAAAGDGAGAGGAGGYGRGAGAGAGAGAAAGAGAGGAGGYGA |
| | | GQGYGAGAGAGAAAGAGAGGAGGYGAGQGYGAGAGAGAAAAA [SEQ |
| | | ID NO: 26] |
| Uloborus diversus | MiSp | GSGAGAGSGYGAGAGAGAGSGYGAGSSASAGSAINTQTVTSSTTTSSQ |
| | | SSAAATGAGYGTGAGTGASAGAAASGAGAGYGGQAGYGQGAGASARAA |
| | | GSGYGAGAGAAAAAGSGYGAGAGAGAGSGYGAGAAA [SEQ ID NO: |
| | | 27] |
| Uloborus diversus | MiSp | GAGAGYRGQAGYIQGAGASAGAAAAGAGVGYGGQAGYGQGAGASAGAA |
| | | AAAGAGAGRQAGYGQGAGASAGAAAAGAGAGRQAGYGQGAGASAGAAA |
| | | AGADAGYGGQAGYGQGAGASAGAAASGAGAGYGGQAGYGQGAGASAGA |
| | | AAAGAGAGYLGQAGYGQGAGASAGAAAGAGAGYGGQAGYGQGTGAAAS |
| | | AAASSA [SEQ ID NO: 28] |
| Araneus | MaSp1 | GGQGGQGGYGGLGSQGAGQGGYGAGQGAAAAAAAAGGAGGAGRGGLGA |
| ventricosus | | GGAGQGYGAGLGGQGGAGQAAAAAAAGGAGGARQGGLGAGGAGQGYGA |
| | | GLGGQGGAGQGGAAAAAAAAGGQGGQGGYGGLGSQGAGQGGYGAGQGG |
| | | AAAAAAAAGGQGGQGGYGGLGSQGAGQGGYGGRQGGAGAAAAAAAA |
| | | [SEQ ID NO: 29] |
| Dolomedes | MaSp1 | GGAGAGQGSYGGQGGYGQGGAGAATATAAAAGGAGSGQGGYGGQGGLG |
| tenebrosus | | GYGQGAGAGAAAAAAAAAGGAGAGQGGYGGQGGQGGYGQGAGAGAAAA |
| | | AAGGAGAGQGGYGGQGGYGQGGGAGAAAAAAAASGGSGSGQGGYGGQG |
| | | GLGGYGQGAGAGAGAAASAAAA [SEQ ID NO: 30] |
| Nephilengys | MaSp | GGAGQGGYGGLGGQGAGAAAAAAGGAGQGGYGGQGAGQGAAAAAASGA |
| cruentata | | GQGGYEGPGAGQGAGAAAAAAGGAGQGGYGGLGGQGAGQGAGAAAAAA |
| | | GGAGQGGYGGLGGQGAGQGAGAAAAAAGGAGQGGYGGQGAGQGAAAAA |
| | | AGGAGQGGYGGLGSGQGGYGRQGAGAAAAAAAA [SEQ ID NO: |
| | | 31] |
| Nephilengys | MaSp | GGAGQGGYGGLGGQGAGAAAAAAGGAGQGGYGGQGAGQGAAAAAASGA |
| cruentata | | GQGGYGGPGAGQGAGAAAAAAGGAGQGGYGGLGGQGAGQGAGAAAAAA |
| | | GGAGQGGYGGQGAGQGAAAAAAGGAGQGGYGGLGSGQGGYGGQGAGAA |
| | | AAAGGAGQGGYGGLGGQGAGQGAGAAAAAA [SEQ ID NO: 32] |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3×FLAG nucleotide coding sequence. In some embodiments, the silk polypeptide nucleotide coding sequence is operatively linked to other affinity tags such as 6-8 His residues (SEQ ID NO: 33).

In some embodiments, the recombinant spider silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the spider silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, wherein the recombinant spider silk protein comprises repeat units wherein each repeat unit has at least 95% sequence identity to a sequence that comprises from 2 to 20 quasi-repeat units; each quasi-repeat unit comprises {GGY-[GPG-X₁]ₙ₁-GPS-(A)ₙ₂} (SEQ ID NO: 34), wherein for each quasi-repeat unit; X₁ is independently selected from the group consisting of SGGQQ (SEQ ID NO: 35), GAGQQ (SEQ ID NO: 36), GQGPY (SEQ ID NO: 37), AGQQ (SEQ ID NO: 38), and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant spider silk polypeptide comprises the polypeptide sequence of SEQ ID NO: 1 (i.e., 18B). In some embodiments, the repeat unit is a polypeptide comprising SEQ ID NO: 2. These sequences are provided in Table 1B:

TABLE 1B
Exemplary polypeptides sequences of recombinant protein and repeat unit

| SEQ ID | Polypeptide Sequence |
|---|---|
| SEQ ID | GGYGPGAGQQGPGSGGQQGPGGQGPYGSGQQGPGGAGQQGPGGQGPYGPGAAAAAAAAAG |
| NO: 1 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAAGGYGPGAGQQGPGSGGQQGPGGQGPYGSGQQGPGGAGQQGPGGQG |
| | PYGPGAAAAAAAAAGGYGPGAGQQGPGGAGQQGPGSQGPGGQGPYGPGAGQQGPGSQGPG |
| | SGGQQGPGGQGPYGPSAAAAAAAAAGGYGPGAGQRSQGPGGQGPYGPGAGQQGPGSQGPG |
| | SGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGP |
| | GSQGPGSGGQQGPGGQGPYGPSAAAAAAAAGGYGPGAGQQGPGSGGQQGPGGQGPYGSGQ |
| | QGPGGAGQQGPGGQGPYGPGAAAAAAAAAGGYGPGAGQQGPGGAGQQGPGSQGPGGQGPY |
| | GPGAGQQGPGSQGPGSGGQQGPGGQGPYGPSAAAAAAAAAGGYGPGAGQRSQGPGGQGPY |
| | GPGAGQQGPGSQGPGSGGQQGPGGQGPYGPSAAAAAAAAGGYGPGAGQQGPGSQGPGSGG |
| | QQGPGGQGPYGPGAAAAAAAVGGYGPGAGQQGPGSQGPGSGGQQGPGGQGPYGPSAAAAA |
| | AAAGGYGPGAGQQGPGSQGPGSGGQQGPGGQGPYGPSAAAAAAAA |
| SEQ ID | GGYGPGAGQQGPGSGGQQGPGGQGPYGSGQQGPGGAGQQGPGGQGPYGPGAAAAAAAAAG |
| NO: 2 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAA |

In some embodiments, the structure of fibers formed from the described recombinant spider silk polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. While not wishing to be bound by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of RPFs. For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities and Degradation

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat-Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include, but are not limited to, water and polyalcohols (polyols) such as glycerol, triglycerol, hexaglycerol, and decaglycerol. Other suitable plasticizers include, but are not limited to, Dimethyl Isosorbite; biasamide of dimethylaminopropyl amine and adiptic acid; 2,2,2-trifluoro ethanol; amide of dimethylaminopropyl amine and caprylic/capric acid; DEA acetamide and any combination thereof. Other suitable plasticizers are discussed in Ullsten et. al, Chapter 5: Plasticizers for Protein Based Materials Viscoelastic and Viscoplastic Materials (2016) (available at https://www.intechopen.com/books/viscoelastic-and-viscoplastic-materials/plasticizers-for-protein-based-materials) and Vierra et al., Natural-based plasticizers and polymer films: A review, European Polymer Journal 47(3):254-63 (2011), the entirely of these are herein incorporated by reference.

As hydrophilic portions of silk polypeptides can bind ambient water present in the air as humidity, water will almost always be present, the bound ambient water may plasticize silk polypeptides. In some embodiments, a suitable plasticizer may be glycerol, present either alone or in combination with water or other plasticizers. Other suitable plasticizers are discussed above.

In addition, in instances where recombinant spider silk polypeptides are produced by fermentation and recovered as recombinant spider silk polypeptide powder from the same, there may be impurities present in the recombinant spider silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant spider silk polypeptide powder or composition. Size Exclusion Chromatography separates molecules based on their relative size and can be used to analyze the relative amounts of recombinant spider silk polypeptide in its full-length polymeric and monomeric forms as well as the amount of high, low and intermediate molecular weight impurities in the recombinant spider silk polypeptide powder. Similarly, Rapid High Performance Liquid Chromatography may be used to measure various compounds present in a solution such as monomeric forms of the recombinant spider silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant spider silk polypeptide may have a purity calculated based on the amount of the recombinant spider silk polypeptide in is monomeric form by weight relative to the other components of the recombinant spider silk polypeptide powder. In various instances, the purity can range from 50% by weight to 90% by weight, depending on the type of recombinant spider silk polypeptide and the techniques used to recover, separate and post-process the recombinant spider silk polypeptide powder.

Both Size Exclusion Chromatography and Reverse Phase High Performance Liquid Chromatography are useful in measuring full-length recombinant spider silk polypeptide, which makes them useful techniques for determining whether processing steps have degraded the recombinant spider silk polypeptide by comparing the amount of full-length spider silk polypeptide in a composition before and after processing. In various embodiments of the present invention, the amount of full-length recombinant spider silk polypeptide present in a composition before and after processing may be subject to minimal degradation. The amount of degradation may be in the range 0.001% by weight to 10% by weight, or 0.01% by weight to 6% by weight, e.g. less than 10% or 8% or 6% by weight, or less than 5% by weight, less than 3% by weight or less than 1% by weight.

### Melt Rheology, Secondary and Tertiary Structures

Rheology is commonly used in fiber spinning to analyze the physio-chemical characteristics of material that is spun into fiber such as polymers. Different rheological characteristics may impact the ability to spin material into fiber and the mechanical characteristics of the spun fiber. Rheology can be also used to indirectly study the secondary and tertiary structures formed by recombinant spider silk polypeptides and/or plasticizer under different pressures, temperatures and conditions. Depending on the embodiment, shear rheometers and/or extensional rheometers may be used to analyze different rheological properties by oscillatory and extensional rheology.

In some embodiments, Capillary Rheometry is used to characterize the glass transition and/or melt transition of compositions comprising recombinant spider silk polypeptide powder and plasticizer. These compositions before being transformed into a melted or flowable state are herein referred to as “recombinant spider silk compositions.” Further, when the recombinant spider silk compositions are in the melted or flowable state, these compositions are herein referred to as “recombinant spider silk melt compositions.”

In some embodiments, the melt transitions and/or glass transitions of the recombinant spider silk compositions can be characterized using a Capillary Rheometer by extruding the recombinant spider silk composition over different ranges of pressures and a “ramp” produced by increasing the shear rate. Depending on the embodiment and instance, the ramp may start at approximately 300 m/s to 1500 m/s. Depending on the embodiment, the pressure may vary from 1 MPa to 125 MPa, often 6 MPa to 50 MPa.

In some embodiments, Differential Scanning calorimetry is used to determine the glass transition and/or melt transition temperature of the recombinant spider silk polypeptide and/or fiber containing the same. In a specific embodiment, Modulated Differential Scanning calorimetry is used to measure the glass transition and/or melt transition temperature.

Depending on the embodiment and the type of recombinant spider silk polypeptide, the glass transition and/or melt transition temperatures may have range of values. However, a measured glass transition and/or melt transition temperature that is much lower than is typically observed for a recombinant spider silk polypeptide in its solid form may indicate that impurities or the presence of other plasticizers.

In addition, Fourier Transform Infrared (FTIR) spectroscopy data may be combined with rheology data to provide both direct characterization of tertiary structures in the recombinant silk powder and/or composition containing the same. FTIR can be used to quantify secondary structures in silk polypeptides and/or composition comprising the silk polypeptides as discussed below in the section entitled “Fourier Transform Infrared (FTIR) Spectroscopy.”

Depending on the embodiment, FTIR may be used to quantify beta-sheet structures present in the recombinant spider silk polypeptide powder and/or composition containing the same. In addition, in some embodiments, FTIR may be used to quantify impurities such as sugars and lipids present in the recombinant spider silk polypeptide powder. However, various chaotropes and solubilizers used in different protein pre-processing methods may diminish the number of tertiary structures in recombinant spider silk polypeptide powder or composition containing the same. Accordingly, there may be no correspondence between the amount of beta sheet structures in recombinant spider silk polypeptide powder before and after it is molded or spun into fiber. Similarly, there may be little to no correspondence between the glass transition temperature of a powder before and after it is molded or spun into fiber.

In some embodiments, rheological data characterizing the recombinant spider silk polypeptides may be combined with FTIR to analyze secondary and tertiary structures formed in the polypeptides. In a specific embodiment, rheological data may be captured in conjunction with FTIR spectra. For exemplary methods of combining rheology and FTIR, see Boulet-Audet et al., Silk protein aggregation kinetics revealed by Rheo-IR, Acta Biomaterialia 10:776-784 (2014), the entirety of which is herein incorporated by reference.

Fourier Transform Infrared (FTIR) spectra can be used to assess the tertiary structure of proteins present in polypeptide powder and/or fibers. Specifically, FTIR spectra can be used to determine the amount of beta sheets present in the fibers that are subject to different spinning and post-processing conditions. Thus, FTIR spectra may be used to determine the relative amount of beta sheet structures based on the different techniques. Alternately, the FTIR spectra may be compared to native insect silk.

Depending on the embodiment, FTIR spectra at different wavenumbers may be used to assess the different tertiary structures present in the fibers. In various embodiments, wavenumbers corresponding to Amide I and Amide II bands may be used to assess various protein structures such as turns, beta-sheets, alpha helices, and side chains. Wavenumbers corresponding to these structures are well known in the art.

In most embodiments, FTIR spectra at wavenumbers corresponding to beta sheets will be used to assess the quantity of beta sheet structures in the polypeptide powder and/or fiber. In a specific embodiment, FTIR spectra at 982-949 cm⁻¹ (CH₂ rocking (A)ₙ), 1695-1690 cm⁻¹ (Amide I) 1620-1625 cm⁻¹ (Amide I), 1440-1445 cm⁻¹ (asymmetric CH₃ bending) and/or 1508 cm⁻¹ (Amide II) are used to determine the amount of beta sheets present. Depending on the embodiment, the different wavenumbers and ranges can be measured to determine the amount of beta sheets present. In some embodiments the FTIR spectra at 982-949 cm⁻¹ is used in order to eliminate interference from corresponding peaks. Exemplary methods of obtaining spectra at these wavenumbers are discussed in detail in Boudet-Audet et al, Identification and classification of silks using infrared spectroscopy, Journal of Experimental Biology, 218:3138-3149 (2015), the entirety of which is herein incorporated by reference.

Similarly, various methods of characterizing impurities in the recombinant silk powder may be combined with rheological and/or FTIR data to analyze the relationship between the presence of impurities and the formation of secondary and/or tertiary structures.

### Recombinant Spider Silk Melt Compositions

It is an object of this invention to make various recombinant spider silk compositions that are capable of being transformed into a melted or flowable state (i.e., capable of being transformed into a recombinant spider silk melt composition) according to the methods described herein. In various embodiments, the concentration of recombinant spider silk polypeptide powder and plasticizer in the composition may be varied based on the properties of the recombinant spider silk polypeptide powder (e.g., the purity of the recombinant spider silk polypeptide powder), the type of plasticizer used, and the desired properties of the fiber. In some embodiments, concentrations may be adjusted based on rheological data such as the data from a Capillary Rheometer.

In some embodiments, a Melt Flow Indexer will be used to determine whether a recombinant spider silk melt composition is capable of being drawn into a fiber. Specifically, a Melt Flow Indexer may be used to measure the ‘melt strength’ of the recombinant spider silk melt composition, or ability to draw the recombinant spider silk melt composition as it is extruded. In various embodiments, concentrations of recombinant spider silk polypeptide and plasticizer may vary based on the desired melt strength.

In some embodiments, various agents may be added to the recombinant spider silk composition to alter the rheological characteristics of the recombinant spider silk composition such as elongational viscosity, shear viscosity and linear viscoelasticity. Suitable agents used to alter the elongational viscosity include polyethylene glycol (PEG), Tween (polysorbate), sodium dodecyl sulfate, polyethylene, or any combination thereof. Other suitable agents are well known in the art.

In some embodiments, a second polymer may be added to create a polymer blend or bi-constituent fiber with the recombinant spider silk composition. In these instances, it may be useful to include a second polymer that has a melting temperature that makes it suitable for melting, in tandem with the recombinant spider silk composition itself, without degrading the amorphous regions of the recombinant spider silk polypeptide. In various embodiments, polymers suitable for blending with recombinant spider silk polypeptides will have a melting temperature (Tm) of less than 200° C., 180° C., 160° C., 140° C., 120° C. or 100° C. Often, the recombinant spider silk polypeptide will have a melting temperature of more than 20° C., or 25° C. or 50° C. A non-limiting list of exemplary polymers and the melting temperatures is included in the table below.

TABLE 1C
Polymers

| Polymer | Tm ° C. |
|---|---|
| LLDPE, Linear Low | 120-130 |
| Density Polyethylene | |
| LDPE, Low Density | 105-120 |
| Polyethylene | |
| MDPE, Medium Density | 120-180 |
| Polyethylene | |
| HDPE, High Density | 130+ |
| Polyethylene | |
| PP, Polypropylene | 130+ |
| PLA PolyLactic Acid | 125-175 |
| EVA Ethyl Vinyl Acetate | 70-85 |
| PBAT Poly(butylene | 110-120 |
| adipate-co-terephthalate) | |
| PBSA Polybutylene | 116 |
| Succinate Adipate | |
| PBS Polybutylene Succinate | 84-115 |
| DuPont ™ Ionomers (e.g. | 80-100 |
| Surlyn ® ionomers) | |
| EPE, Expanded Polyethylene | 126 |
| PC Polycarbonate | 155 |
| PCL Polycaprolactone | 60 |

Depending on the embodiment, suitable concentrations of recombinant spider silk polypeptide powder by weight in the recombinant spider silk composition ranges from: 1 to 90% by weight, 3 to 80% by weight, 5 to 70% by weight, 10 to 60% by weight, 15 to 50% by weight, 18 to 45% by weight, or 20 to 41% by weight.

In the instance where glycerol is used as a plasticizer, suitable concentration of glycerol by weight in the recombinant spider silk composition ranges from: 1 to 60% by weight, 10 to 60% by weight, 10 to 50% by weight, 10 to 40% by weight, 15 to 40% by weight, 10 to 30% by weight, or 15 to 30% by weight.

In the instance where water is used as a plasticizer, a suitable concentration of water by weight in the recombinant spider silk composition ranges from: 5 to 80% by weight, 15 to 70% by weight, 20 to 60% by weight, 25 to 50% by weight, 19 to 43% by weight, or 19 to 27% by weight. Where water is used in combination with another plasticizer, it may be present in the range 5 to 50% by weight, 15 to 43% by weight or 19 to 27% by weight.

In some embodiments, water may be evaporated during extrusion and/or cooling process depending the treatment and/or the die size used. In some embodiments, water loss after molding may range from 1 to 50% by weight, 3 to 40% weight, 5 to 30% weight, 7 to 20% weight, 8 to 18% weight, or 10-15% based on the total water amount. Often loss will be less than 15%, in some cases less than 10%, for instance 1 to 10% by weight. Evaporation may be intentional or as a result of the treatment applied. The degree of evaporation can be easily controlled, for instance by selection of operating temperatures, flow rates and pressures applied, as would be understood in the art.

In some embodiments, suitable plasticizers may include polyols (e.g., glycerol), water, lactic acid, methyl hydroperoxide, ascorbic acid, 1,4-dihydroxybenzene (1,4 benzenediol) benzene-1,4-diol, phosphoric acid, ethylene glycol, propylene glycol, triethanolamine, acid acetate, propane-1,3-diol or any combination thereof.

In various embodiments, the amount of plasticizer can vary according to the purity and relative composition of the recombinant spider silk polypeptide powder. For example, a higher purity powder may have less impurities such as a low molecular weight compounds that may act as plasticizers and therefore require the addition of a higher percentage by weight of plasticizer.

In specific embodiments, various ratios (by weight) of the plasticizer (e.g. a combination of glycerol and water) to the recombinant spider silk polypeptide powder may range from 0.5 or 0.75 to 350% by weight plasticizer: recombinant spider silk polypeptide powder, 1 or 5 to 300% by weight plasticizer: recombinant spider silk polypeptide powder, 10 to 300% by weight plasticizer: recombinant spider silk polypeptide powder, 30 to 250% by weight plasticizer: recombinant spider silk polypeptide powder, 50 to 220% by weight plasticizer: recombinant spider silk protein, 70 to 200% by weight plasticizer: recombinant spider silk polypeptide powder, or 90 to 180% by weight plasticizer: recombinant spider silk polypeptide powder. As used herein, reference to 0.5 to 350% by weight plasticizer: recombinant spider silk polypeptide powder corresponds to a ratio of 0.5:1 to 350:1.

Without intending to be limited by theory, in various embodiments of the present invention, inducing the recombinant spider silk composition to transition into a flowable state (e.g. inducing a recombinant spider silk melt composition) may be used as a pre-processing step in any formulation in circumstances where it is beneficial to include the recombinant spider silk polypeptide in its monomeric form. More specifically, inducing the recombinant spider silk melt composition may be used in applications where it is desirable to prevent the aggregation of the monomeric recombinant spider silk polypeptide into its crystalline polymeric form or to control the transition of the recombinant spider silk polypeptide into its crystalline polymeric form at a later stage in processing. In one specific embodiment, the recombinant spider silk melt composition may be used to prevent aggregation of the recombinant spider silk polypeptide prior to blending the recombinant spider silk polypeptide with a second polymer. In another specific embodiment, the recombinant spider silk melt composition may be used to create a base for a cosmetic or skincare product where the recombinant spider silk polypeptide is present in the base in its monomeric form. In this embodiment, having the recombinant spider silk polypeptide in its monomeric form in a base allows for the controlled aggregation of the monomer into its crystalline polymeric form upon contact with skin or through various other chemical reactions

### Inducing a Melt or Flowable State

According some embodiments of the present invention, the recombinant spider silk composition is transformed into melted or flowable state through the application of shear force and/or pressure, typically both. Suitable means for generating a combination of shear force and pressure include but are not limited to: single screw extruders, twin screw extruders, melt flow extruders, and capillary rheometers.

In some embodiments, a twin screw extruder is used to provide the necessary pressure and shear force to transform the recombinant spider silk composition into a melted or flowable composition. In some embodiments, the twin screw extruder is configured to provide a shear force ranging from: 1.5 Newton meters (Nm) to 13 Newton meters, 2 Newton meters to 10 Newton meters, 2 Newton meters to 8 Newton meters, or 2 Newton meters to 6 Newton meters. In some embodiments, the shear force provided by the twin screw extruder depends, in part, on the rotations per minute of the twin screw extruder. In various embodiments and configurations the rotations per minute (RPMs) of the twin screw extruder may range from 10 RPMs to 300 RPMs. In various embodiments, the twin screw extruder is configured to provide a pressure ranging from 1 MPa to 300 MPa in conjunction with the shear force.

In optional embodiments, the twin screw extruder is configured to apply heat to the recombinant spider silk composition before and/or after it is transformed into a recombinant spider silk melt composition. In some embodiments, the barrel of the twin screw extruder (i.e. the cylinder in which the twin screws mix a composition) is subject to heating. In other embodiments, a portion of the twin screw extruder proximal to a spinneret (i.e. orifice through which the recombinant spider silk melt composition is extruded) is subject to heating. Alternatively, no heat is applied, the melt/flowable state being induced entirely through heat generated from the shearing forced applied to the recombinant spider silk composition in the twin screw extruder. For example, in some embodiments, the amount of heat applied to obtain a melt/flowable state would be similar to equal to ambient room temperature (e.g. approximately than 20° C.).

In various embodiments, the temperature to which the recombinant spider silk melt composition is heated will be minimized in order to minimize or entirely prevent degradation of the recombinant spider silk polypeptide. In specific embodiments, the recombinant spider silk melt will be heated to a temperature of less than 120° C., less than 100° C., less than 80° C., less than 60° C., less than 40° C., or less than 20° C. Often the melt will be at a temperature in the range 10° C. to 120° C., 10° C. to 100° C., 15° C. to 80° C., 15° C. to 60° C., 18° C. to 40° C. or 20±2° C. during processing.

In other embodiments, other devices may be used to provide pressure and shear force necessary to transform the recombinant spider silk composition into a melted or flowable state. As discussed above, a capillary rheometer may also be used to provide the necessary shear force and pressure to transform the recombinant spider silk composition into a flowable or melted state.

In some embodiments, the recombinant spider silk composition is optionally heated after it is in a melted or flowable state and/or prior to extrusion of the melted or flowable recombinant spider silk melt composition. Where heating is required, perhaps because the recombinant spider silk composition is of high glass transition temperature, the device used to provide shear force and pressure to transform the recombinant spider silk composition into a melted or flowable state may be coupled, either directly or indirectly to a heated extrusion device. In a specific embodiment, a twin screw cylinder mixer is coupled (either directly or indirectly) to a heated extrusion device. Depending on the embodiment and configuration of the heated extrusion device, the heated extrusion device may be maintained at temperatures ranging from 20 to 120° C., 80 to 110° C., 85 to 100° C., 85 to 95° C. and/or 90 to 95° C.

The extruded recombinant spider silk melt composition is herein referred to as a recombinant spider silk extrudate. Depending on the application of the recombinant spider silk extrudate, the spinneret through which the extrudate is extruded may vary in diameter. For example, in embodiments where the recombinant spider silk extrudate is extruded into a mold to form a molded object, the spinneret may have a diameter greater than 200 mm, greater than 150 mm, greater than 100 mm, greater than 50 mm for instance in the range 100 mm to 500 mm, 150 mm to 400 mm or 200 mm to 300 mm. As discussed below, in some embodiments the recombinant spider silk extrudate can be processed into pellets that may be re-processed by again subjecting the pellets to shear force and pressure sufficient to transform the spider silk extrudate into a recombinant spider silk melt composition. In embodiments where the recombinant spider silk extrudate is processed into pellets, the spinneret may have a diameter greater than 2 mm, greater than 1.5 mm or greater than 1 mm, for instance, the diameter may be in the range 1 mm to 5 mm, 1.5 mm to 4 mm, or 2 mm to 3 mm.

In embodiments where the recombinant spider silk extrudate is made into a fiber, the spinneret may have an orifice that is less than 500 μm (for instance in the range 10 μm to 500 μm). Depending on the required initial denier of the extruded fiber, the recombinant spider silk protein melt composition may be extruded through spinnerets with varying orifice sizes. In specific embodiments, the orifice may range from 25 μm to 500 μm, 50 μm to 250 μm, or 75 μm to 125 μm. In some embodiments, the ideal orifice size will be based on the final draw ratio of the fiber. For example, a higher initial denier of an extruded fiber may be subject to a higher draw ratio.

In most embodiments of the present invention, both the recombinant spider silk melt composition and the recombinant spider silk extrudate will be substantially homogeneous meaning that the material, as inspected by light microscopy, does not have any inclusions or precipitates. In some embodiments, light microscopy may be used to measure birefringence which can be used as a proxy for alignment of the recombinant spider silk into a three-dimensional lattice. Birefringence is the optical property of a material having a refractive index that depends on the polarization and propagation of light. Specifically, a high degree of axial order as measured by birefringence can be linked to high tensile strength. In some embodiments, recombinant spider silk melt extrudate will have minimal birefringence.

According to the present invention, a homogeneous flowable state can be induced through the application of shear force and pressure only, although optionally heat may be applied. The combination of shear force and pressure alone, without the application of heat or with optional heat, has been found to provide compositions which do not degrade during processing of the recombinant spider silk polypeptide in the recombinant spider silk melt composition and the recombinant spider silk extrudate. This is desirable and beneficial as retaining the full length recombinant spider silk polypeptide in the extrudate composition produces optimal material properties, such as crystallinity, resulting in higher quality products. In embodiments of the present invention, the recombinant spider silk melt extrudate achieved from the application of shear force and pressure (and optionally heat) has minimal or negligible degradation.

The amount of degradation of the recombinant spider silk polypeptide may be measured using various techniques. As discussed above, the amount of degradation of the recombinant spider silk polypeptide may be measured using Size Exclusion Chromatography to measure the amount of full-length recombinant spider silk polypeptide present. In various embodiments, the composition is degraded in an amount of less than 6.0 weight % after it is formed into a molded body. In another embodiment, the composition is degraded in an amount of less than 4.0 weight % after molding, less than 3.0 weight %, less than 2.0 weight %, or less than 1.0 weight % (such that the amount of degradation may be in the range 0.001% by weight to 10%, 8%, 6%, 4%, 3%, 2% or 1% by weight, or 0.01% by weight to 6%, 4%, 3%, 2% or 1% by weight). In another embodiment, the recombinant spider silk protein in the extrudate and/or melt composition is substantially non-degraded.

### Drawing Fiber

Where the extrudate is being used in fiber formation, precursor fiber may be drawn in order to increase the orientation of the fiber and promote three-dimensional crystalline structure. The application of force in drawing promotes molecules to align on the axis of the fiber. Polymeric molecules such as polypeptides are partially aligned when forced to flow through the spinneret hole. The fibers may be hand drawn or machine drawn. Hand drawing will often offer well aligned fibers with low birefringence yet with minimal reduction in fiber diameter.

In the present invention, the alignment may be optimized by passing the precursor fiber over a uniform hot surface while the fiber is drawn. The term “hot surface” as used herein refers to a surface that provides both a substantially uniform heat and a substantially uniform surface. Using a hot surface as a heat source eliminates variability seen using ambient heat sources, resulting in greater uniformity in results and consequent scalability of the process for commercial mass production of the fiber. In some embodiments, the hot surface will be a metal bar or other metal surface. In other embodiments, the hot surface may be made of ceramic or other materials. Depending on the embodiment, the hot surface can be curved or otherwise configured to facilitate the fiber moving over the hot surface.

In embodiments of the present invention, the undrawn extruded fiber may be simultaneously moved over the hot surface as it is drawn. Depending on the embodiment, the temperature of the hot surface can range from 160 to 210° C., 180 to 210° C., 190 to 210° C., 195 to 210° C., 195 to 205° C., or 200 to 205° C.

Depending on the embodiment, the undrawn extruded fiber can be subject to different draw ratios while it is drawn over the hot surface. Depending on the embodiment, the draw ratio may range from 2 to 7. In some embodiments, the maximum stable draw ratio may depend on the temperature of the hot surface.

In some embodiments, the temperature of the hot surface is calculated as a function of the glass transition temperature of the undrawn extruded fiber. For example, the temperature of the hot surface can be calculated to be greater than 5° C., 10° C., 15° C., 20° C., or 25° C. greater than the glass transition temperature of the recombinant silk protein powder and/or the undrawn extruded fiber. In other words, in the range 0, or 0.1° C. to 25° C. greater than the glass transition temperature of the recombinant silk protein powder, often in the range 0 to 10° C., 15° C., 20° C. greater.

Depending on the embodiment and the rate at which fiber is passed over the uniform hot surface (referred to herein as the “reel rate”), the hot surface can vary in length (i.e. the size in cm of the hot surface that the fiber is drawn over), thus changing the duration of time that the undrawn extruded fiber is subject to heat and deformation. In most embodiments, the width of the hot bar will be no less than 1 cm. However, in various embodiments the width of the hot surface can range from 1 to 50 cm, 1 to 2 cm, 1 to 3 cm, 1 to 5 cm, 5 to 38 cm, 38 to 50 cm. Depending on the embodiment, the reel rate can range from 1 to 60 meters a minute.

Depending on the reel rate and the length of the hot surface, the total residence time over the hot surface may vary. In most embodiments the total residence time can range from 0.2 seconds to 3 seconds.

In addition, the undrawn fiber may be subject to varying force which provides different draw ratios. In most embodiments, the tensile force will be provided by godets. In some embodiments, the godets will be placed such that the fiber that is passed over the hot surface is at an angle relative to the hot surface. For example, in instances where the hot surface is curved, the godets may be placed such that the fiber that is passed over the hot surface is at an angle of 10 to 40 degrees relative to the hot surface.

In various embodiments, the deformation rate (i.e., the amount of deformation that the fiber is subject to with heat and drawing) of the undrawn fiber can vary based on the above factors. Deformation rate may be calculated based on the rate that the undrawn fiber is fed to the hot surface and the rate that the fiber is collected from the hot surface. For example, the fiber may be fed to the hot surface at a rate of 1 meters/minute and collected from the hot surface at a rate of 5 meters/minute. In a specific embodiment, the deformation rate is calculated using the following equation, where the rate that the fiber is fed to the hot surface is represented v₁, the rate that the fiber is collected from the hot surface is v₂ and the length the deformation takes place over is L₀:

$$\dot{ϵ} ( t ) = \frac{{v}_{2} - {v}_{1}}{{L}_{0}} \qquad Equation 1$$

Depending on the embodiment, drawing over a hot surface may be performed in one step or multiple (i.e. two, three, or four) steps. Parameters such as the strain rate, the deformation rate, the reel rate, the temperature of the hot surface and the length of the hot surface may be varied or otherwise different at each step. Performing drawing over multiple steps may affect the overall strain rate of the fiber, which may enhance formation of crystalline beta-sheet structures, often improving fiber strength.

### Post-Processing Fiber

Various methods of post-processing may be employed to improve the molecular alignment of the fiber. Depending on the amount of plasticizer and/or the recombinant spider silk present in fiber, the fiber may be heat treated (e.g. annealed using steam or heat). In other instances, the fiber may be treated with various solvents to anneal the fiber and improve crystallinity of the protein (for instance 18B protein) in the fiber. In some instances, the fiber may be annealed using an alcohol such as methanol. In a specific embodiment, the fiber may be annealed using alcohol vapor.

In some instances, treating a fiber or a textile with one or more conditioners, lubricants, surfactants, emulsifiers, anti-cohesion agents or annealing agents before treating the fiber with water will alter the hand feel or drape of a textile after treatment with water. In a specific embodiment cyclopentasiloxane or PDMS are used as conditioners. In a specific embodiment, annealing a fiber or a textile formed from a fiber with an alcohol improves the hand feel and drape of a water-treated fiber or textile.

### Re-Melting and Re-extruding Extrudate

In some embodiments of the present invention, the process for preparing the recombinant spider silk extrudate may additionally comprise re-processing a molded body comprising the recombinant spider silk extrudate (e.g. a pellet, fiber or other molded article formed from recombinant spider silk extrudate). In these embodiments, the recombinant spider silk extrudate is subject to sufficient shear force and pressure to transform the recombinant spider silk extrudate into a melted or flowable state.

Without intending to be limited by theory, subjecting the recombinant spider silk polypeptide to shear force and pressure in the presence of a plasticizer such as glycerol converts the recombinant spider silk polypeptide into an “open-form recombinant spider silk polypeptide” in which the recombinant spider silk polypeptide unfolds and forms interactions with the glycerol. Due to the interactions with glycerol, this “open-form recombinant spider silk polypeptide” forms less intermolecular and intramolecular beta-sheet interactions. Specifically, the open form recombinant spider silk polypeptide is prevented from forming intermolecular interactions to form an irreversible three-dimensional lattice.

Because there is minimal degradation (if any) of the recombinant spider silk polypeptide during the melting and extruding process, the recombinant spider silk extrudate may be transformed back into a recombinant spider silk melt composition and re-extruded any number of times. In this sense, the composition is “thermoplastic”, as it may be heated, allowed to cool and harden many times without significant degradation of the protein or the composition. In various embodiments, the recombinant spider silk extrudate may be re-melted and re-extruded at least 20 times, at least 10 times, or at least 5 times. In these embodiments, the degradation seen over multiple re-melting and re-extruding steps may be as low as 10%. The option of re-extrusion without degradation allows for the production of substantially homogeneous compositions, and also for the repurposing or redesign of products formed from the composition. For instance, molded products which are of insufficient quality, may be re-extruded and remolded. End of life product recycling is also a possibility.

### EXAMPLES

### Example 1: Purity of Recombinant 18B Polypeptide Powder

Recombinant spider silk—18B polypeptide sequences (SEQ ID NO: 1) comprising the FLAG tag—were produced through various lots of large-scale fermentation, recovered and dried in powders (“18B powder”). Reverse Phase High Performance Liquid Chromatography (“RP-HPLC”) was used to measure the amount by weight of 18B polypeptide monomer in the powder. The samples were dissolved using a 5M Guanidine Thiocyanate (GdSCN) reagent and injected onto an Agilent Poroshell 300SB C3 2.1×75 mm 5 μm column to separate constituents on the basis of hydrophobicity. The detection modality was UV absorbance of peptide bond at 215 nm (360 nm reference). The sample concentration of 18B-FLAG monomer was determined by comparison with an 18B-FLAG powder standard, for which the 18B-FLAG monomer concentration had been previously determined using Size Exclusion Chromatography (SEC-HPLC)

The sample powder was found to include 57.964 Mass % of 18B monomer.

### Example 2: Generating Recombinant Silk Powder Extrudates

The recombinant silk powder of Example 1 was mixed using a household spice grinder. Ratios of water and glycerol were added to the recombinant silk powder (“18B powder”) to generate recombinant spider silk compositions with different ratios of protein powder to plasticizer as tabulated below in Table 2.

Batches of 10 to 100 grams of the recombinant spider silk compositions (i.e., “formulations”) listed below in Table 2 were mixed using a Xceptional Instruments Twin Screw Extruder (TSE) (item number TT-ZE5-MSMS-3HT) which was used for all TSE experiments. The stainless steel (S316) extruder barrel had 3 heating zones ˜5 cm in length each. The screws used were a standard pair of stainless steel (S316) co-rotating screws 180 mm in length and 9 mm in diameter and (L/D ratio of 20:1). The screws had a pitch of 9 mm.

For the P25W05G70, P49W21G30 and P65W20G15 formulations listed below, recombinant spider silk compositions were first extruded into pellets that were re-processed in the following experiments by re-extruding the pellets. To make pellets, recombinant spider silk compositions comprising 18B/Water/Glycerol mixtures were introduced to the TSE using a metallic funnel and pushed into contact with the twin screws using a tamping device continuously for several minutes while the TSE was running at 300 RPM with a temperature of ˜90-95° C. across all three barrel regions including the start, middle and end barrel regions. The material was extruded in the melt state (i.e., as a recombinant spider silk melt composition) through a 0.5 mm die whose orifice was at a 180° angle to the screw axis to form a recombinant spider silk extrudate.

The 0.5 mm recombinant spider silk extrudates emerged from the die as continuous, elastomeric “noodles” ˜>10 meters in length. Pellets were generated by sequentially placing 5-10 g quantities of corresponding extrudates compositions into a kitchen spice grinder and subjecting them to 5 second pulses for a total of 6 pulses (30 seconds total). The pellets were inspected to ensure they had lengths of no more than 5 mm, with average lengths of pellets being about 2.5 mm.

For the P71W19G10 formulation listed below, the 18B/water/glycerol recombinant spider silk mixture was pre-mixed and extruded directly (i.e. without first extruding as a pellet) under the conditions described in Example 2 to form recombinant spider silk extrudate.

TABLE 2
Recombinant Spider Silk Formulations Composition by Weight

| | 18 B | Water | Glycerol |
|---|---|---|---|
| | Powder % | % by | % |
| Formulation | by weight | weight | by weight |
| P25W05G70 | 25% | 5% | 70% |
| P49W21G30 | 49% | 21% | 30% |
| P65W20G15 | 65% | 20% | 15% |
| P71W19G10 | 71% | 19% | 10% |

### Example 3: Generating Recombinant Silk Extrudates With Minimal Degradation

To assess degradation over a number of different conditions, the recombinant spider silk formulations listed in Example 2 were subject to various temperatures during extrusion and various amounts of pressure and shear force. Specifically, the rotations per minute of the twin screw extruded pellets were varied to provide a variable amount of torque and shear force. Various temperature and RPM combinations used to transform the recombinant spider silk formulation into the melt state and extrude the different samples are included below.

The extruded pellets of the P49W21G30 and P65W20G15 formulation listed in Table 1 were again subject to extrusion at various RPM and temperatures using the Xceptional Instruments TSE. Other parameters for operating the Xceptional Instruments TSE were the same as those described above with respect to Example 2.

As described in Example 2, the P71W19G10 formulation was also extruded at various RPM and temperatures using the Xceptional Instruments TSE. Other parameters for operating the Xceptional Instruments TSE were the same as those described above with respect to Example 2.

Data characterizing the relative amounts of high, low and intermediate molecular weight impurities, monomeric 18B and aggregate 18B was collected using Size Exclusion Chromatography (SEC) as follows: 18B powder was dissolved in 5M Guanidine Thiocyanate and injected onto a Yarra SEC-3000 SEC-HPLC column to separate constituents on the basis of molecular weight. Refractive index was used as the detection modality. 18B aggregates, 18B monomer, low molecular weight (1-8 kDa) impurities, intermediate molecular weight impurities (8-50 kDa) and high molecular weight impurities (110-150 kDa) were quantified. Relevant composition was reported as mass % and area %. BSA was used as a general protein standard with the assumption that >90% of all proteins demonstrate dn/dc values (the response factor of refractive index) within ˜7% of each other. Poly (ethylene oxide) was used as a retention time standard, and a BSA calibrator was used as a check standard to ensure consistent performance of the method.

Tables 3-5 below lists the various SEC analyses for the extrudates produced under various RPMs and temperatures. The fifth column includes either the difference in 18B monomer (area %) reported in the starting pellets and extrudates (P49W21G30 and P65W20G15) or the difference in 18B monomer (area %) reported in the starting powder and extrudates (P71W19G10). FIGS. 1-3 are described in detail below and include graphs corresponding to Tables 3-5, respectively. From these it can be seen that degradation is minimal across all temperatures and RPMs tested, indicating a flexibility of processing conditions and a general robustness to processing using extrusion methods.

TABLE 3
SEC analysis for P49W21G30

| | | | | Difference between | | | |
|---|---|---|---|---|---|---|---|
| | | | | 18B monomer % | | | |
| | | | 18B | starting pellets | High | Int. | Low |
| Sample ID | Temp. | RPM | monomer % | and samples | MW | MW | MW |
| P49W21G30-1 | 20° C. | 10 | 48.4 | 10.91 | 1.55 | 33.17 | 10.88 |
| P49W21G30-2 | 20° C. | 100 | 42.53 | 16.78 | 1.81 | 35.82 | 14.14 |
| P49W21G30-3 | 20° C. | 200 | 47.77 | 11.54 | 3.55 | 31.28 | 10.73 |
| P49W21G30-4 | 20° C. | 300 | 43.52 | 15.79 | 1.46 | 35.46 | 14.75 |
| P49W21G30-5 | 40° C. | 10 | 54.78 | 4.53 | 4.69 | 27.53 | 4.2 |
| P49W21G30-6 | 40° C. | 100 | 56.87 | 2.44 | 4.82 | 26.18 | 3.07 |
| P49W21G30-7 | 40° C. | 200 | 53.65 | 5.66 | 4.11 | 27.83 | 6 |
| P49W21G30-8 | 40° C. | 300 | 55.15 | 4.16 | 4.70 | 26.75 | 5.66 |
| P49W21G30-9 | 60° C. | 10 | 52.06 | 7.25 | 4.32 | 28.68 | 7.08 |
| P49W21G30-10 | 60° C. | 100 | 54.46 | 4.85 | 4.27 | 28.65 | 4.93 |
| P49W21G30-11 | 60° C. | 200 | 55.74 | 3.57 | 4.31 | 27.61 | 4.18 |
| P49W21G30-12 | 60° C. | 300 | 54.21 | 5.1 | 3.71 | 28.56 | 4.72 |
| P49W21G30-13 | 80° C. | 10 | 53.78 | 5.53 | 3.73 | 29.2 | 5.19 |
| P49W21G30-14 | 80° C. | 100 | 55.97 | 3.34 | 3.53 | 26.32 | 6.36 |
| P49W21G30-15 | 80° C. | 200 | 53.94 | 5.37 | 3.77 | 28.69 | 5.58 |
| P49W21G30-16 | 80° C. | 300 | 54.02 | 5.29 | 3.50 | 27.65 | 6.99 |
| P49W21G30-17 | 95° C. | 10 | 45.16 | 14.15 | 3.58 | 34.9 | 8.18 |
| P49W21G30-18 | 95° C. | 100 | 55.76 | 3.55 | 2.25 | 28.98 | 5.4 |
| P49W21G30-19 | 95° C. | 200 | 50.2 | 9.11 | 2.17 | 30.64 | 10.53 |
| P49W21G30-20 | 95° C. | 300 | 46.31 | 13 | 2.72 | 32.65 | 11.55 |
| P49W21G30-21 | 120° C. | 10 | 53.91 | 5.4 | 3.68 | 28.35 | 5.88 |
| P49W21G30-22 | 120° C. | 100 | 52.11 | 7.2 | 3.97 | 31.65 | 6.19 |
| P49W21G30-23 | 120° C. | 200 | 48.85 | 10.46 | 2.89 | 31.83 | 10.15 |
| P49W21G30-24 | 120° C. | 300 | 51.09 | 8.22 | 3.51 | 31.37 | 7.8 |

TABLE 4
SEC analysis for P65W20G15

| | | | | Difference between | | | |
|---|---|---|---|---|---|---|---|
| | | | | 18B monomer % | | | |
| | | | 18B | in samples and | High | Int. | Low |
| Sample ID | Temp. | RPM | monomer % | starting pellets | MW | MW | MW |
| P65W20G15-1 | 20° C. | 10 | 53.58 | 5.73 | 3.368 | 30.29 | 4.23 |
| P65W20G15-2 | 20° C. | 100 | 53.76 | 5.55 | 3.514 | 28.89 | 6.17 |
| P65W20G15-3 | 20° C. | 200 | 53 | 6.31 | 3.272 | 30.55 | 5.3 |
| P65W20G15-4 | 20° C. | 300 | 52.62 | 6.69 | 3.558 | 30.28 | 5.63 |
| P65W20G15-5 | 40° C. | 10 | 54.35 | 4.96 | 3.186 | 30.3 | 4.88 |
| P65W20G15-6 | 40° C. | 100 | 53.68 | 5.63 | 4.279 | 27.96 | 4.32 |
| P65W20G15-7 | 40° C. | 200 | 54.13 | 5.18 | 3.462 | 28.44 | 5.48 |
| P65W20G15-8 | 40° C. | 300 | 52.01 | 7.3 | 3.933 | 30.01 | 6.11 |
| P65W20G15-9 | 60° C. | 10 | 55.78 | 3.53 | 3.332 | 27.92 | 5.03 |
| P65W20G15-10 | 60° C. | 100 | 58.05 | 1.26 | 3.814 | 26.08 | 3.55 |
| P65W20G15-11 | 60° C. | 200 | 57.47 | 1.84 | 3.308 | 27.06 | 4.25 |
| P65W20G15-12 | 60° C. | 300 | 58.55 | 0.76 | 2.874 | 26.54 | 3.9 |
| P65W20G15-13 | 95° C. | 10 | 52.02 | 7.29 | 2.47 | 29.51 | 8.32 |
| P65W20G15-14 | 95° C. | 100 | 49.92 | 9.39 | 2.48 | 29.3 | 11.24 |
| P65W20G15-15 | 95° C. | 200 | 44.02 | 15.29 | 1.96 | 32.37 | 15 |
| P65W20G15-16 | 95° C. | 300 | 51.31 | 8 | 1.84 | 31.52 | 8.22 |
| P65W20G15-17 | 140° C. | 10 | 50.49 | 8.82 | 5.53 | 28.04 | 4.6 |
| P65W20G15-18 | 140° C. | 100 | 59.4 | −0.09 | 3.241 | 24.7 | 3.4 |
| P65W20G15-19 | 140° C. | 200 | 54.96 | 4.35 | 4.245 | 27.17 | 3.78 |
| P65W20G15-20 | 140° C. | 300 | 54.85 | 4.46 | 4.353 | 26.14 | 5.12 |

TABLE 5
SEC analysis for P71W19G10

| | | | | Difference between | | | |
|---|---|---|---|---|---|---|---|
| | | | | 18B monomer % | | | |
| | | | 18B | in samples and | High | Int. | Low |
| Sample ID | Temp. | RPM | monomer % | starting powder | MW | MW | MW |
| P71W19G10-1 | 90° C. | 10 | 48.61 | 10.7 | 2.90 | 29.95 | 11.01 |
| P71W19G10-2 | 90° C. | 100 | 55.17 | 4.14 | 2.47 | 28.87 | 5.64 |
| P71W19G10.5-3 | 90° C. | 200 | 42.27 | 17.04 | 3.44 | 34.84 | 11.89 |
| P71W19G10-4 | 90° C. | 300 | 31.41 | 27.9 | 4.02 | 39.24 | 17.53 |
| P71W19G10-5 | 120° C. | 10 | 37.23 | 22.08 | 4.32 | 38.32 | 7.73 |
| P71W19G10-6 | 120° C. | 100 | 33.1 | 26.21 | 5.42 | 38.23 | 8.74 |
| P71W19G10-7 | 120° C. | 200 | 32.61 | 26.7 | 5.01 | 38.46 | 11.38 |
| P71W19G10-8 | 120° C. | 300 | 49.58 | 9.73 | 2.20 | 32.5 | 8.72 |

FIG. 1 shows SEC data for P49W21G30 samples listed above in Table 3 under extrusion conditions at 20, 40, 60, 80, 95 or 120° C., where extrudates were obtained for each temperature using operating parameters of 10, 100, 200 or 300 RPM. 18B monomers (black bars), intermediate molecular weight impurities (grey bars) and low molecular weight impurities (cross hatched bars) are shown as area %.

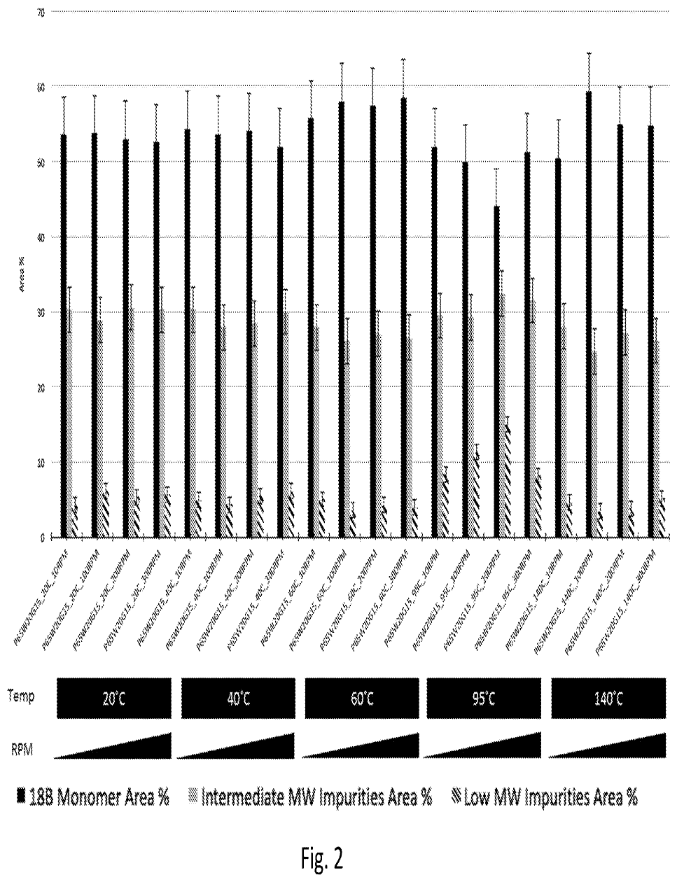

FIG. 2 shows SEC data for P65W20G15 samples listed above in Table 4 under extrusion conditions at 20, 40, 60, 95 or 140° C., where extrudates were obtained for each temperature using operating parameters of 10, 100, 200 or 300 RPM. 18B monomers (black bars), intermediate molecular weight impurities (grey bars) and low molecular weight impurities (cross hatched bars) are shown as area %.

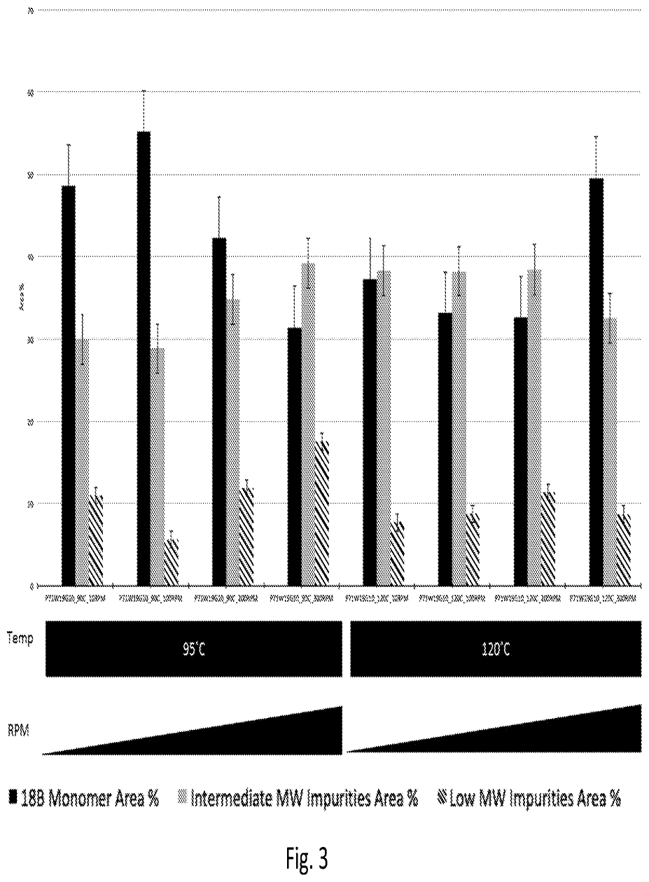

FIG. 3 shows SEC data for P71W19G10 samples listed above in Table 5 under extrusion conditions at 90 or 120° C., where extrudates were obtained for each temperature using operating parameters of 10, 100, 200 or 300 RPM. 18B monomers (black bars), intermediate molecular weight impurities (grey bars) and low molecular weight impurities (cross hatched bars) are shown as area %.

### Example 4: Thermogravimetric Analysis-P49W21G30

In order to analyze water loss during extrusion, the water content of the recombinant spider silk compositions before extrusion and the recombinant spider silk extrudates after extrusion was analyzed by TGA (thermogravimetric analysis) using a TA brand TGA Q500 instrument. For the P49W21G30 and P65W20G15 samples, the water content of the pellets used for the extrusion experiments described in Example 3 was used as a reference sample to measure water loss. For the P71W19G10 samples, the water content of the recombinant spider silk compositions used for the extrusion experiments described in Example 3 was used as a reference sample to measure water loss.

For each sample, 10 mg, +/−1 mg of powders or pellets comprising the formulations listed above were analyzed. To measure water content, samples were run “in air” as opposed to “in nitrogen.” Samples were sequentially introduced into the TGA furnace using the equipped autosampler. The temperature was programmed to increase at a rate of 20° C./minute from room temperature, until it reached 110° C. using the TA brand software suite. The samples were then kept at this temperature for 45 minutes. The samples were then removed from the furnace, and the furnace was flushed with air for 15 minutes before starting the next run.

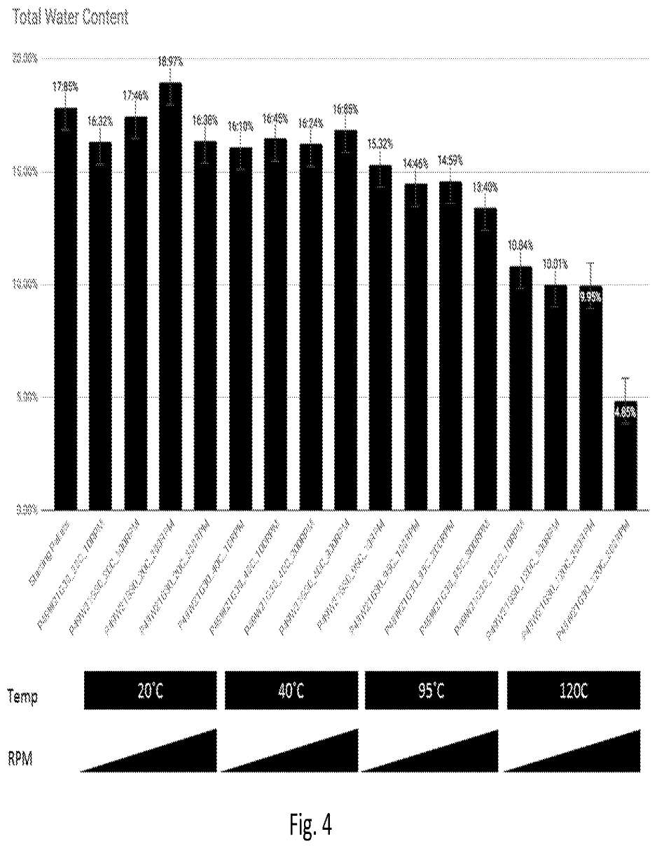

Tables 6-8 below lists the various measurements for the reference samples (i.e. starting pellets or powder) and the extruded samples. FIGS. 4-6 include graphs of the data included in Tables 6-8, respectively. From this data it can be seen that water loss during extrusion is low, and well within acceptable limits for an extrusion process. Typically water loss is in the range 2-18%.

TABLE 6
Water loss in P49W21G30

| | | | Water in | Water | |
|---|---|---|---|---|---|
| | | | Starting | In | |
| Sample ID | Temp. | RPM | Pellets | Extrudates | Δ Water |
| P49W21G30-1 | 20° C. | 10 | 17.95% | 16.32% | 1.63% |
| P49W21G30-2 | 20° C. | 100 | 17.95% | 17.46% | 0.49% |
| P49W21G30-4 | 20° C. | 300 | 17.95% | 16.38% | 1.57% |
| P49W21G30-5 | 40° C. | 10 | 17.95% | 16.10% | 1.85% |
| P49W21G30-6 | 40° C. | 100 | 17.95% | 16.45% | 1.50% |
| P49W21G30-7 | 40° C. | 200 | 17.95% | 16.24% | 1.71% |
| P49W21G30-8 | 40° C. | 300 | 17.95% | 16.85% | 1.10% |
| P49W21G30-9 | 60° C. | 10 | 17.95% | 8.22% | 9.73% |
| P49W21G30-10 | 60° C. | 100 | 17.95% | 11.93% | 6.02% |
| P49W21G30-11 | 60° C. | 200 | 17.95% | 10.59% | 7.36% |
| P49W21G30-12 | 60° C. | 300 | 17.95% | 9.92% | 8.04% |
| P49W21G30-13 | 80° C. | 10 | 17.95% | 9.18% | 8.77% |
| P49W21G30-14 | 80° C. | 100 | 17.95% | 9.08% | 8.87% |
| P49W21G30-15 | 80° C. | 200 | 17.95% | 8.63% | 9.32% |
| P49W21G30-16 | 80° C. | 300 | 17.95% | 8.82% | 9.14% |
| P49W21G30-17 | 95° C. | 10 | 17.95% | 15.32% | 2.63% |
| P49W21G30-18 | 95° C. | 100 | 17.95% | 14.46% | 3.49% |
| P49W21G30-19 | 95° C. | 200 | 17.95% | 14.59% | 3.36% |
| P49W21G30-20 | 95° C. | 300 | 17.95% | 13.40% | 4.55% |
| P49W21G30-21 | 120° C. | 10 | 17.95% | 10.84% | 7.11% |
| P49W21G30-22 | 120° C. | 100 | 17.95% | 10.01% | 7.94% |
| P49W21G30-23 | 120° C. | 200 | 17.95% | 9.95% | 8.00% |
| P49W21G30-24 | 120° C. | 300 | 17.95% | 4.85% | 13.10% |

TABLE 7
Water loss in P65W20G15

| | | | Water in | Water | |
|---|---|---|---|---|---|
| | | | Starting | In | |
| Sample ID | Temp. | RPM | Pellets | Extrudates | Δ Water |
| P65W20G15-1 | 20° C. | 10 | 11.63% | 8.79% | 2.84% |
| P65W20G15-2 | 20° C. | 100 | 11.63% | 8.08% | 3.55% |
| P65W20G15-3 | 20° C. | 200 | 11.63% | 7.78% | 3.85% |
| P65W20G15-4 | 20° C. | 300 | 11.63% | 7.43% | 4.20% |
| P65W20G15-5 | 40° C. | 10 | 11.63% | 7.34% | 4.30% |
| P65W20G15-6 | 40° C. | 100 | 11.63% | 7.07% | 4.56% |
| P65W20G15-7 | 40° C. | 200 | 11.63% | 7.20% | 4.43% |
| P65W20G15-8 | 40° C. | 300 | 11.63% | 7.10% | 4.53% |
| P65W20G15-9 | 60° C. | 10 | 11.63% | 7.17% | 4.46% |
| P65W20G15-10 | 60° C. | 100 | 11.63% | 6.82% | 4.81% |
| P65W20G15-11 | 60° C. | 200 | 11.63% | 6.81% | 4.82% |
| P65W20G15-12 | 60° C. | 300 | 11.63% | 6.47% | 5.16% |
| P65W20G15-16 | 95° C. | 300 | 11.63% | 11.43% | 0.20% |
| P65W20G15-17 | 140° C. | 10 | 11.63% | 6.83% | 4.80% |
| P65W20G15-18 | 140° C. | 100 | 11.63% | 6.22% | 5.41% |

TABLE 8
Water loss in P71W19G10

| | | | Water in | Water | |
|---|---|---|---|---|---|
| | | | Starting | In | |
| Sample ID | Temp. | RPM | Powder | Extrudates | Δ Water |
| P71W19G10-1 | 90° C. | 10 | 7.22% | 7.16% | 0.06% |
| P71W19G10-2 | 90° C. | 100 | 7.22% | 6.84% | 0.38% |
| P71W19G10-3 | 90° C. | 200 | 7.22% | 6.81% | 0.41% |
| P71W19G10-4 | 90° C. | 300 | 7.22% | 6.79% | 0.43% |
| P71W19G10-5 | 120° C. | 10 | 7.22% | 6.21% | 1.01% |
| P71W19G10-6 | 120° C. | 100 | 7.22% | 6.08% | 1.15% |
| P71W19G10-7 | 120° C. | 200 | 7.22% | 5.94% | 1.28% |

FIG. 4 shows TGA data for samples listed above in Table 6 which were generated under extrusion conditions at 20, 40, 95 and 120° C., where extrudates were obtained for each temperature using operating parameters of 10, 100, 200 and 300 RPM. FIG. 4 also shows TGA data for a reference sample of the starting pellets used to generate these samples. The data show % water content of the samples across all treatments, with water loss ranging from ˜1-13% when compared to starting pellets.

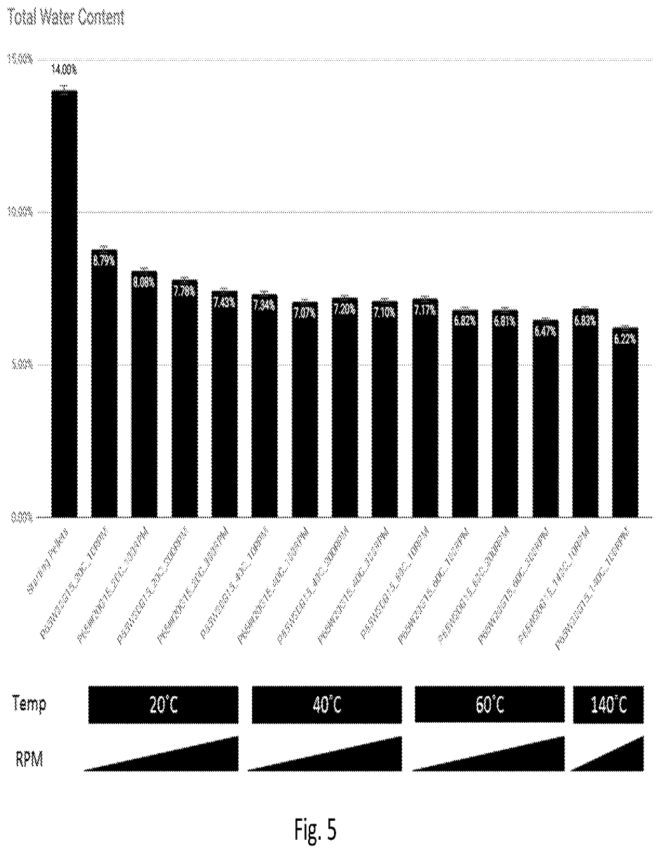

FIG. 5 shows TGA data for samples listed above in Table 7 which were generated under extrusion conditions at 20, 40, 60 and 140° C., where extrudates were obtained for each temperature using operating parameters of 10, 100, 200 and 300 RPM. FIG. 5 also shows TGA data for a reference sample of the starting pellets used to generate these samples. The data show % water content of the samples across all treatments, with water loss ranging from ˜1-8% when compared to starting pellets.

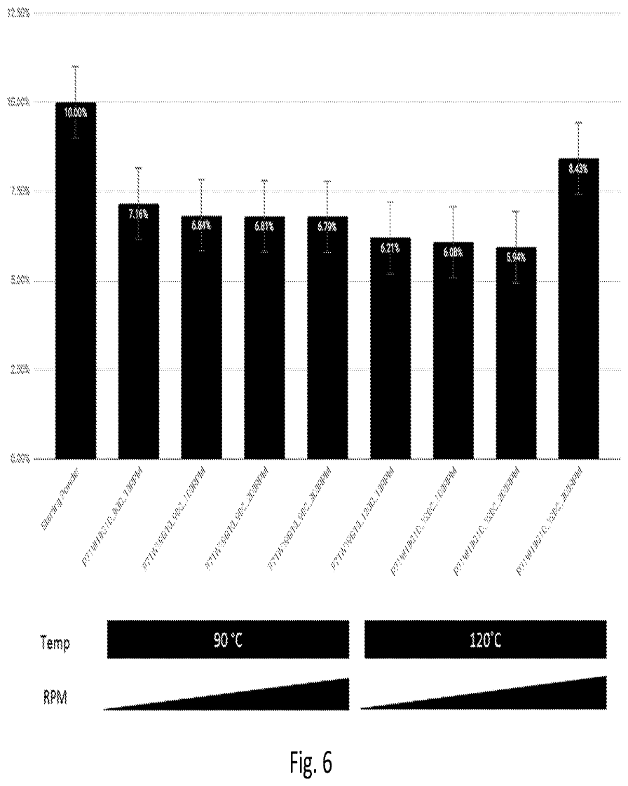

FIG. 6 shows TGA data for samples listed above in Table 8 which were generated under extrusion conditions at 90 and 120° C., where extrudates were obtained for each temperature using operating parameters of 10, 100, 200 and 300 RPM. FIG. 5 also shows TGA data for a reference sample of the starting powder used to generate these samples. The data show % water content of the samples across all treatments, with water loss ranging from ˜1.5-4% when compared to starting powder.

### Example 5: Beta Sheet Content Analysis Using Fourier Transform Infrared Spectroscopy

To assess the formation of secondary and tertiary structures in the extrudates, the beta-sheet content was measured by FTIR (Fourier Transform infrared spectroscopy). FTIR was performed on the extrudates using Bruker Alpha spectrometer equipped with a diamond attenuated total reflection accessory preceded by a wire grid polarizer selecting mostly S (perpendicular) polarized light. Recombinant polypeptide powder and the precursor fiber were included as controls. To quantify the molecular alignment three spectra of each orientation (0 and 90° relative to the polarization electric field) were collected with 32 scans at 4 cm⁻¹ resolution from 4000 to 600 cm⁻¹.

The average values for the peak corresponding to 982-949 cm⁻¹ were calculated based on the following steps. Absorbance values were offset by subtracting the average between 1900 and 1800 cm⁻¹ without bands. Spectra were then normalized by dividing the average between 1350 and 1315 cm⁻¹ corresponding to the isotropic (non-oriented) side chain vibration bands. The beta-sheet content metric was taken to be the average of the integrated absorbance values between 982 and 949 cm⁻¹.

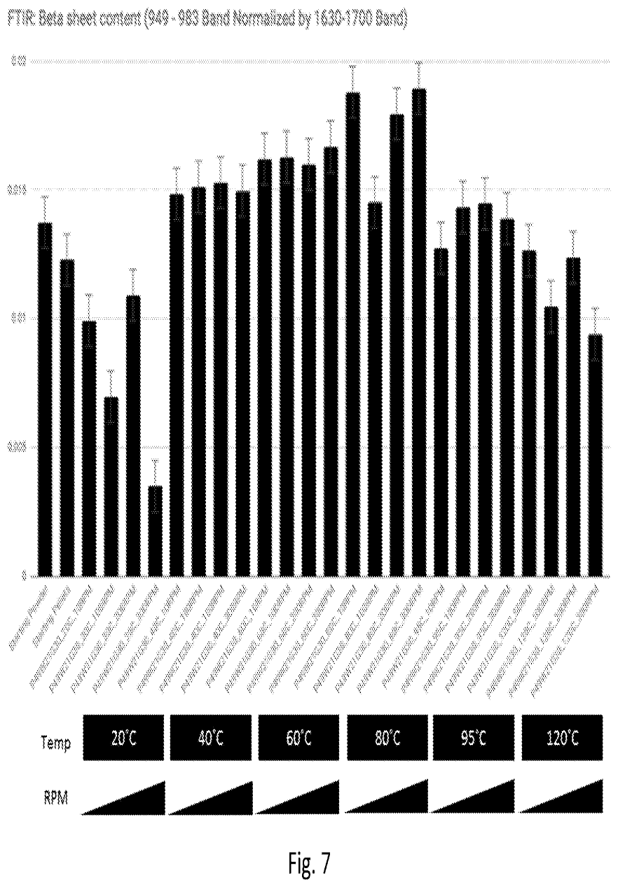

The beta sheet content of the recombinant spider silk extrudates (i.e., “Sample Beta Sheets”) were compared to i) the beta sheet content in the starting recombinant spider silk polypeptide powder used to generate the recombinant spider silk compositions (i.e., “Reference Pre-hydrated Powder”), and ii) the beta sheet content in the starting pellets (P49W21G30 and P65W20G15) (i.e., “Reference Pellets”) Tables 9-11 below lists the measurements for the reference samples and the extrudates produced under the conditions tabulated below. FIGS. 7-9 include graphs of the data shown in Tables 9-11. As can be seen, there is no significant change in the beta-sheet content of the materials from starting recombinant silk polypeptide powder to recombinant spider silk extrudate, indicating that this method enables plasticization and mobility of the amorphous protein domains without disruption to the beta-sheets as would be the case if solvent processing were used.

TABLE 9
Beta Sheet Formation in P49W21G30

| | | | Reference | | |
|---|---|---|---|---|---|
| | | | Pre- | | |
| | | | hydrated | Reference | Sample |
| | | | Powder | Pellets | Beta |
| | | | Beta Sheets | Beta Sheets | Sheets |
| | | | ~982- | ~982- | ~982- |
| Sample ID | Temp. | RPM | 949 nm | 949 nm | 949 nm |
| P49W21G30-1 | 20° C. | 10 | 0.01194 | .01229 | 0.009923 |
| P49W21G30-2 | 20° C. | 100 | 0.01194 | .01229 | 0.006975 |
| P49W21G30-3 | 20° C. | 200 | 0.01194 | .01229 | 0.010909 |
| P49W21G30-4 | 20° C. | 300 | 0.01194 | .01229 | 0.003502 |
| P49W21G30-5 | 40° C. | 10 | 0.01194 | .01229 | 0.014843 |
| P49W21G30-6 | 40° C. | 100 | 0.01194 | .01229 | 0.015117 |
| P49W21G30-7 | 40° C. | 200 | 0.01194 | .01229 | 0.015277 |
| P49W21G30-8 | 40° C. | 300 | 0.01194 | .01229 | 0.014973 |
| P49W21G30-9 | 60° C. | 10 | 0.01194 | .01229 | 0.016206 |
| P49W21G30-10 | 60° C. | 100 | 0.01194 | .01229 | 0.016281 |
| P49W21G30-11 | 60° C. | 200 | 0.01194 | .01229 | 0.015997 |
| P49W21G30-12 | 60° C. | 300 | 0.01194 | .01229 | 0.016674 |
| P49W21G30-13 | 80° C. | 10 | 0.01194 | .01229 | 0.018788 |
| P49W21G30-14 | 80° C. | 100 | 0.01194 | .01229 | 0.014512 |
| P49W21G30-15 | 80° C. | 200 | 0.01194 | .01229 | 0.017957 |
| P49W21G30-16 | 80° C. | 300 | 0.01194 | .01229 | 0.018933 |
| P49W21G30-17 | 95° C. | 10 | 0.01194 | .01229 | 0.012738 |
| P49W21G30-18 | 95° C. | 100 | 0.01194 | .01229 | 0.014334 |
| P49W21G30-19 | 95° C. | 200 | 0.01194 | .01229 | 0.014475 |
| P49W21G30-20 | 95° C. | 300 | 0.01194 | .01229 | 0.013899 |
| P49W21G30-21 | 120° C. | 10 | 0.01194 | .01229 | 0.012653 |
| P49W21G30-22 | 120° C. | 100 | 0.01194 | .01229 | 0.010467 |
| P49W21G30-23 | 120° C. | 200 | 0.01194 | .01229 | 0.012384 |
| P49W21G30-24 | 120° C. | 300 | 0.01194 | .01229 | 0.009402 |

TABLE 10
Beta Sheet Formation in P65W20G15

| | | | Reference | Reference | Sample |
|---|---|---|---|---|---|
| | | | Powder | Pellets | Beta |
| | | | Beta Sheets | Beta Sheets | Sheets |
| | | | ~982- | ~982- | ~982- |
| Sample ID | Temp. | RPM | 949 nm | 949 nm | 949 nm |
| P65W20G15-1 | 20° C. | 10 | 0.02411 | .01719 | 0.01802 |
| P65W20G15-2 | 20° C. | 100 | 0.02411 | .01719 | 0.02023 |
| P65W20G15-3 | 20° C. | 200 | 0.02411 | .01719 | 0.02022 |
| P65W20G15-4 | 20° C. | 300 | 0.02411 | .01719 | 0.01838 |
| P65W20G15-5 | 40° C. | 10 | 0.02411 | .01719 | 0.02021 |
| P65W20G15-6 | 40° C. | 100 | 0.02411 | .01719 | 0.01945 |
| P65W20G15-7 | 40° C. | 200 | 0.02411 | .01719 | 0.01955 |
| P65W20G15-8 | 40° C. | 300 | 0.02411 | .01719 | 0.02083 |
| P65W20G15-9 | 60° C. | 10 | 0.02411 | .01719 | 0.02292 |
| P65W20G15-10 | 60° C. | 100 | 0.02411 | .01719 | 0.01776 |
| P65W20G15-11 | 60° C. | 200 | 0.02411 | .01719 | 0.01926 |
| P65W20G15-12 | 60° C. | 300 | 0.02411 | .01719 | 0.01924 |
| P65W20G15-13 | 95° C. | 10 | 0.02411 | .01719 | 0.01971 |
| P65W20G15-14 | 95° C. | 100 | 0.02411 | .01719 | 0.01905 |
| P65W20G15-15 | 95° C. | 200 | 0.02411 | .01719 | 0.01980 |
| P65W20G15-16 | 95° C. | 300 | 0.02411 | .01719 | 0.02094 |
| P65W20G15-17 | 140° C. | 10 | 0.02411 | .01719 | 0.01956 |
| P65W20G15-18 | 140° C. | 100 | 0.02411 | .01719 | 0.01936 |
| P65W20G15-19 | 140° C. | 200 | 0.02411 | .01719 | 0.01914 |
| P65W20G15-20 | 140° C. | 300 | 0.02411 | .01719 | 0.01863 |

TABLE 11
Beta Sheet Formation in P71W19G10

| | | | Reference Powder | Sample Beta |
|---|---|---|---|---|
| | | | Beta Sheets | Sheets |
| Sample ID | Temp. | RPM | ~982-949 nm | ~982-949 nm |
| P71W19G10-1 | 90° C. | 10 | 0.02411 | 0.02174 |
| P71W19G10-2 | 90° C. | 100 | 0.02411 | 0.01889 |
| P71W19G10-3 | 90° C. | 200 | 0.02411 | 0.02161 |
| P71W19G10-4 | 90° C. | 300 | 0.02411 | 0.01925 |
| P71W19G10-5 | 120° C. | 10 | 0.02411 | 0.02113 |
| P71W19G10-6 | 120° C. | 100 | 0.02411 | 0.02329 |
| P71W19G10-7 | 120° C. | 200 | 0.02411 | 0.02258 |
| P71W19G10-8 | 120° C. | 300 | 0.02411 | 0.02107 |

FIG. 7 shows FTIR data for samples listed above in Table 9 generated under extrusion conditions at 20, 40, 60, 80, 95 or 120° C., where extrudates were obtained for each temperature using operating parameters of 10, 100, 200 or 300 RPM. The data was extracted from the 949-982 and show no clear trends compared to starting pellets.

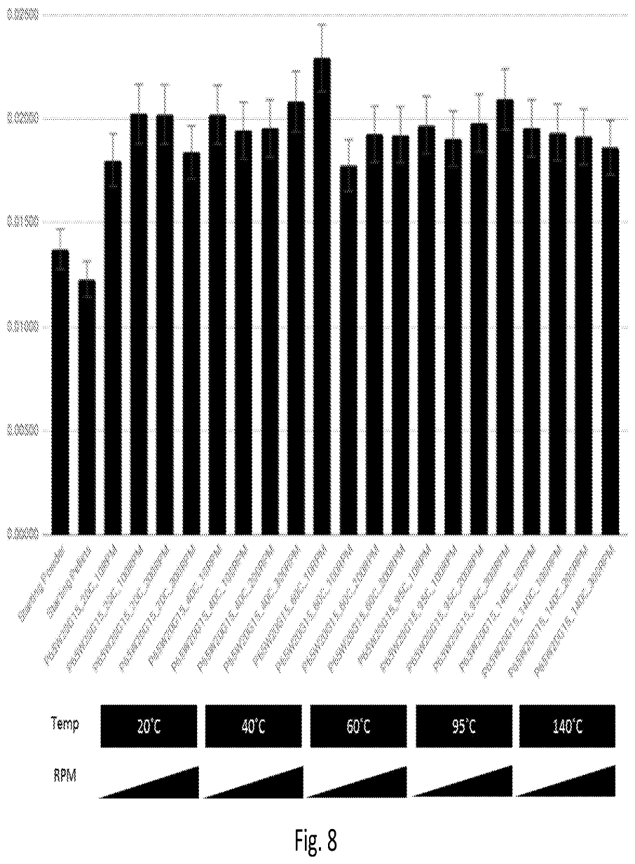

FIG. 8 shows FTIR data for samples for samples listed above in Table 10 which were generated under extrusion conditions at 20, 40, 60, 95 or 140° C., where extrudates were obtained for each temperature using operating parameters of 10, 100, 200 or 300 RPM. The data was extracted from the 949-982 band and show no clear trends compared to starting pellets

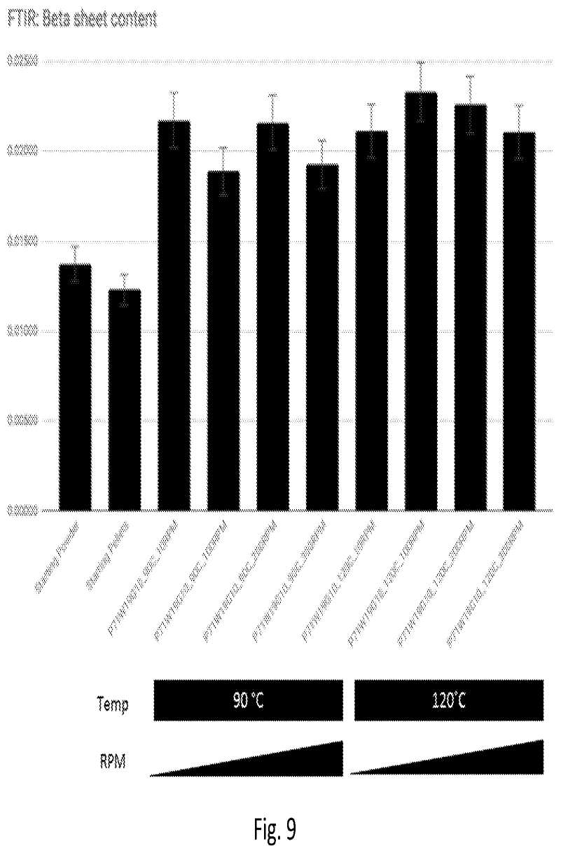

FIG. 9 shows FTIR data for samples for samples listed above in Table 11 which were generated under extrusion conditions at 90 or 120° C., where extrudates were obtained for each temperature using operating parameters of 10, 100, 200 or 300 RPM. The data was extracted from the 949-982 band to avoid artifacts incurred by the presence of water, and show no clear trends compared to starting pellets.

### Example 6: Polarized Light Microscopy

Polarized Light Microscopy (PL) was used to examine the smoothness and homogeneity of the various extrudates. Light and Polarized Light (PL) images were obtained using a Leica DM750P polarized light microscope, using a 4×PL objective. The Microscope was coupled to the complementary PC based image analysis Leica Application Suite, LAS V4.9. ˜20-30 mm long TSE extrudates were carefully placed along the long axis of standard microscope slides and placed horizontally (East-West; i.e. 0°) above the microscope aperture. Sample edges were initially brought into focus, followed by overall focusing of the sample. The samples were initially viewed under white light, controlled by the illumination control knob, and images captured with the appropriate scale bars included. In all cases the auto-brightness feature of the LAS V4.9 software was switched to off.

Next, the Analyzer/Bertrand Lens module was engaged by flipping the lower rocker of the module to the right (the “A” position/Analyzer in), while ensuring the upper rocker of the Analyzer/Bertrand Lens Module was flipped to the left (the “O” position/Bertrand Lens out). This set up allows for analysis in “cross-polarization mode” which is a state of optical alignment in which the allowed oscillatory directions of the light passing through the polarizer and analyzer are oriented at 90°.

In order to control for background fluctuations in light intensity, all samples were initially viewed, and the brightness of the background was reduced with the illumination control knob until it just reached complete blackness. Each of the eyepieces was then covered with an eyepiece light-blocking accessory to prevent ambient light from passing through during the image capture sequence. Images were captured using the LAS V4.9 software package at 0° and 45° orientations. The 45° images where obtained by rotating the glass side to a 45° angle using the circular rotating stage that this microscope is equipped with.

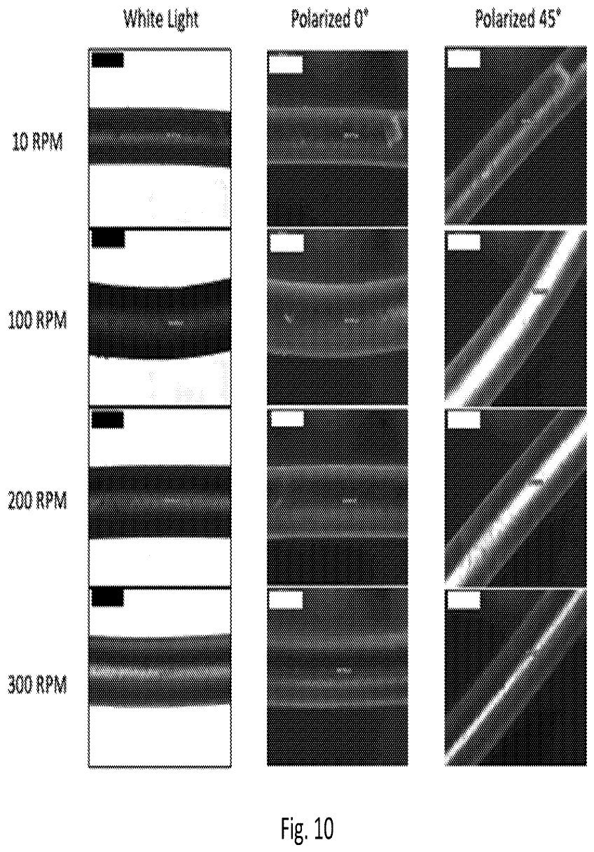

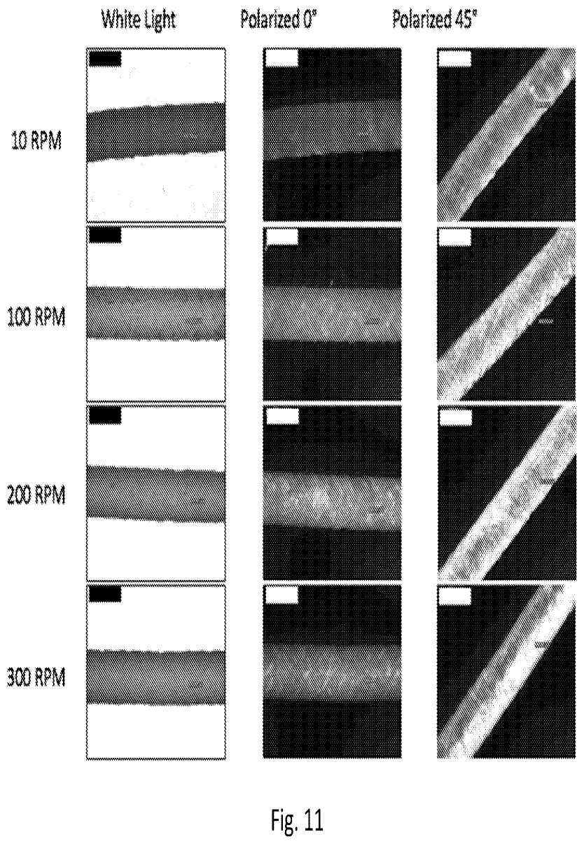

FIGS. 10 and 11 are images of the exemplary samples captured using polarized light microscopy. These show that fibers that are smooth with low melt fracture can be obtained using the claimed processes. Conditions are therefore clearly suitable for melt flow and extrusion. In addition, under many conditions qualitative birefringence was observed, as was axial alignment.

FIG. 10 shows pictures produced from samples P49W21G30-1, P49W21G30-2, P49W21G30-3 and P49W21G30-4 all of which were produced at 20° C. with varying RPMS. Under these conditions the extrudates were smooth with low melt fracture. Polarized Light Microscopy shows preferential axial alignment depending on conditions (examine 45° for differences), where 100 RPM yielded the greatest axial alignment.

FIG. 11 shows pictures produced from samples P49W21G30-17, P49W21G30-18, P49W21G30-19 and P49W21G30-20 all of which were produced at 95° C. with varying RPMS. The extrudates showed moderate melt fracture/surface imperfections. Polarized Light Microscopy showed an increase in axial alignment from 10-100 RPM. From 100-300 RPM the samples showed similar distinction to one another when examined at 0 and 45°.

### Example 7: Metabolites Analysis of Glycerol Content

In order to determine the loss of glycerol from the recombinant spider silk composition during extrusion, the glycerol content was analyzed using a Benson Polymeric 150×7.8 mm H+ 7110-0 HPLC column equipped with a Phenomenex Security Guard Carbo H+ Guard Column, was used with a mobile phase of 0.004 M sulfuric acid. Glycerol calibrants were initially run to enable quantitation. In order to measure the amount of glycerol in the 18B based samples, glycerol present in the compositions was measured before (i.e. as pellets or powder) and after extrusion. For each sample, 25 mg of powder or pellets was dissolved in 1 ml of 0.004 M Sulfuric Acid, and sonicated for 1 hr. The samples were then vortexed and placed in HPLC vials for subsequent runs for each condition/treatment.

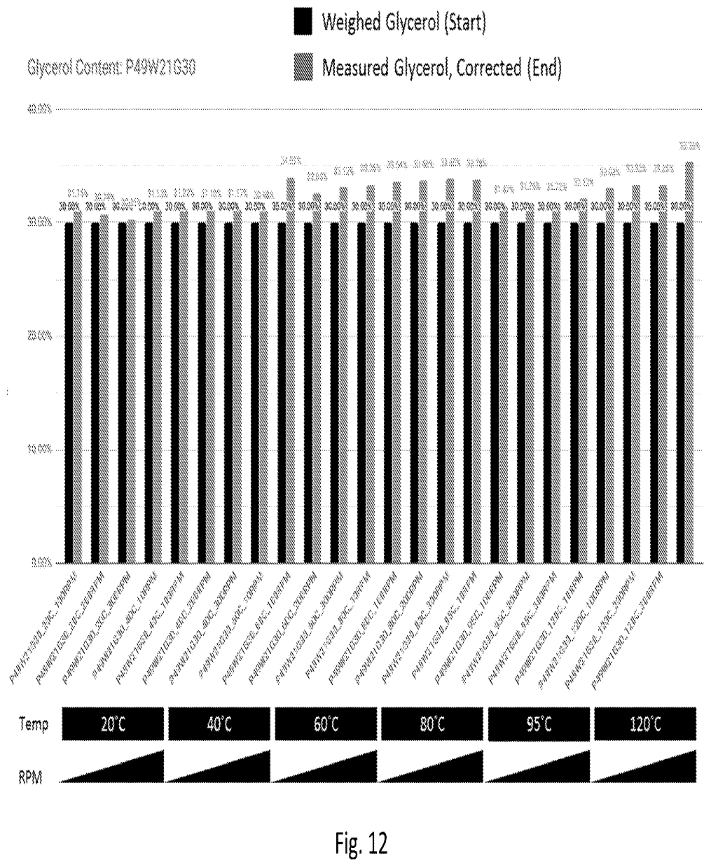

Tables 12-14 below list the various measurements for the extrudates produced under the conditions tabulated below. FIGS. 12-14 include graphs of the same samples. From these it can be seen that glycerol content in the compositions is stable across the range of conditions tested, as evidenced by minimal loss during testing.

TABLE 12
Glycerol Loss in Extrudates - P49W21G30

| | | | | Glycerol | | |
|---|---|---|---|---|---|---|
| | | | Weighed | Concentration, | Measured | |
| | | | Glycerol | corrected for | Glycerol | Δ |
| Sample ID | Temp. | RPM | Wt. % | water loss | Concentration | Glycerol |
| P49W21G30-1 | 20° C. | 10 | 30% | 31.15% | 38.99% | 1.15% |
| P49W21G30-2 | 20° C. | 100 | 30% | 30.78% | 39.14% | 0.78% |
| P49W21G30-3 | 20° C. | 200 | 30% | 30.31% | 39.28% | 0.31% |
| P49W21G30-4 | 20° C. | 300 | 30% | 31.13% | 39.37% | 1.13% |
| P49W21G30-5 | 40° C. | 10 | 30% | 31.22% | 32.74% | 1.22% |
| P49W21G30-6 | 40° C. | 100 | 30% | 31.10% | 33.16% | 1.10% |
| P49W21G30-7 | 40° C. | 200 | 30% | 31.17% | 32.90% | 1.17% |
| P49W21G30-8 | 40° C. | 300 | 30% | 30.98% | 32.90% | 0.98% |
| P49W21G30-9 | 60° C. | 10 | 30% | 34.01% | 32.87% | 4.01% |
| P49W21G30-10 | 60° C. | 100 | 30% | 32.63% | 33.36% | 2.63% |
| P49W21G30-11 | 60° C. | 200 | 30% | 33.12% | 32.90% | 3.12% |
| P49W21G30-12 | 60° C. | 300 | 30% | 33.36% | 33.23% | 3.36% |
| P49W21G30-13 | 80° C. | 10 | 30% | 33.64% | 33.29% | 3.64% |
| P49W21G30-14 | 80° C. | 100 | 30% | 33.68% | 33.65% | 3.68% |
| P49W21G30-15 | 80° C. | 200 | 30% | 33.85% | 34.24% | 3.85% |
| P49W21G30-16 | 80° C. | 300 | 30% | 33.78% | 33.44% | 3.78% |
| P49W21G30-17 | 95° C. | 10 | 30% | 31.47% | 39.85% | 1.47% |
| P49W21G30-18 | 95° C. | 100 | 30% | 31.76% | 39.99% | 1.76% |
| P49W21G30-19 | 95° C. | 200 | 30% | 31.72% | 39.65% | 1.72% |
| P49W21G30-20 | 95° C. | 300 | 30% | 32.12% | 40.28% | 2.12% |
| P49W21G30-21 | 100° C. | 10 | 30% | 33.03% | 33.44% | 3.03% |
| P49W21G30-22 | 100° C. | 100 | 30% | 33.33% | 34.22% | 3.33% |
| P49W21G30-23 | 100° C. | 200 | 30% | 33.35% | 34.94% | 3.35% |
| P49W21G30-24 | 100° C. | 300 | 30% | 35.36% | 34.72% | 5.36% |

TABLE 13
Glycerol Loss in Extrudates - P65W20G15

| | | | | Glycerol | | |
|---|---|---|---|---|---|---|
| | | | Weighed | Concentration, | Measured | |
| | | | Glycerol | corrected for | Glycerol | Δ |
| Sample ID | Temp. | RPM | Wt. % | water loss | Concentration | Glycerol |
| P65W20G15-1 | 20° C. | 10 | 15% | 16.89% | 16.88% | 1.89% |
| P65W20G15-2 | 20° C. | 100 | 15% | 17.03% | 16.77% | 2.03% |
| P65W20G15-3 | 20° C. | 200 | 15% | 17.09% | 16.97% | 2.09% |
| P65W20G15-4 | 20° C. | 300 | 15% | 17.16% | 16.88% | 2.16% |
| P65W20G15-5 | 40° C. | 10 | 15% | 17.18% | 17.26% | 2.18% |
| P65W20G15-6 | 40° C. | 100 | 15% | 17.23% | 17.17% | 2.23% |
| P65W20G15-7 | 40° C. | 200 | 15% | 17.20% | 17.44% | 2.20% |
| P65W20G15-8 | 40° C. | 300 | 15% | 17.22% | 17.55% | 2.22% |
| P65W20G15-9 | 60° C. | 10 | 15% | 17.21% | 17.61% | 2.21% |
| P65W20G15-10 | 60° C. | 100 | 15% | 17.28% | 17.48% | 2.28% |
| P65W20G15-11 | 60° C. | 200 | 15% | 17.28% | 17.69% | 2.28% |
| P65W20G15-12 | 60° C. | 300 | 15% | 17.35% | 17.57% | 2.35% |
| P65W20G15-13 | 95° C. | 10 | 15% | 15.66% | 21.73% | 0.66% |
| P65W20G15-14 | 95° C. | 100 | 15% | 15.66% | 20.53% | 0.66% |
| P65W20G15-15 | 95° C. | 200 | 15% | 15.72% | 20.29% | 0.72% |
| P65W20G15-16 | 95° C. | 300 | 15% | 16.41% | 21.43% | 1.41% |
| P65W20G15-17 | 140° C. | 10 | 15% | 17.27% | 18.06% | 2.27% |
| P65W20G15-18 | 140° C. | 100 | 15% | 17.40% | 18.00% | 2.40% |
| P65W20G15-19 | 140° C. | 200 | 15% | 16.04% | 18.04% | 1.04% |
| P65W20G15-20 | 140° C. | 300 | 15% | 16.13% | 18.37% | 1.13% |

TABLE 14
Glycerol Loss in Extrudates - P71W19G10

| | | | | Glycerol | | |
|---|---|---|---|---|---|---|
| | | | Weighed | Concentration, | Measured | |
| | | | Glycerol | corrected for | Glycerol | Δ |
| Sample ID | Temp. | RPM | Wt. % | water loss | Concentration | Glycerol |
| P71W19G10-1 | 90° C. | 10 | 10% | 10.82% | 13.86% | 0.82% |
| P71W19G10-2 | 90° C. | 100 | 10% | 10.76% | 13.83% | 0.76% |
| P71W19G10-3 | 90° C. | 200 | 10% | 10.87% | 14.07% | 0.87% |
| P71W19G10-4 | 90° C. | 300 | 10% | 9.58% | 14.09% | −0.42%* |
| P71W19G10-5 | 120° C. | 10 | 10% | 9.63% | 13.62% | −0.37%* |
| P71W19G10-6 | 120° C. | 100 | 10% | 9.58% | 13.64% | −0.42%* |
| P71W19G10-7 | 120° C. | 200 | 10% | 10.14% | 13.68% | 0.14% |
| P71W19G10-8 | 120° C. | 300 | 10% | 10.91% | 14.44% | 0.91% |

*Result within error range of testing instrument.

FIG. 12 shows Metabolites data for samples listed above in Table 12 generated under extrusion conditions at 20, 40, 60, 80, 95 and 120° C., where extrudates were obtained for each temperature using operating parameters of 10, 100, 200 and 300 RPM. Glycerol loss was negligible across all treatments.

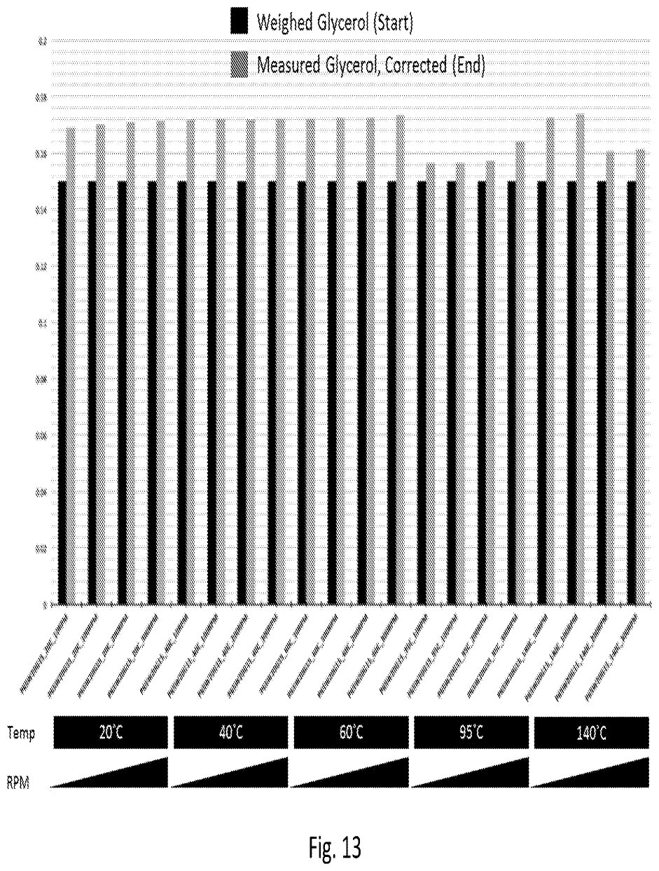

FIG. 13 shows Metabolites data for samples listed above in Table 13 generated under extrusion conditions at 20, 40, 60, 95 and 140° C., where extrudates were obtained for each temperature using operating parameters of 10, 100, 200 and 300 RPM. Glycerol loss was negligible across all treatments.

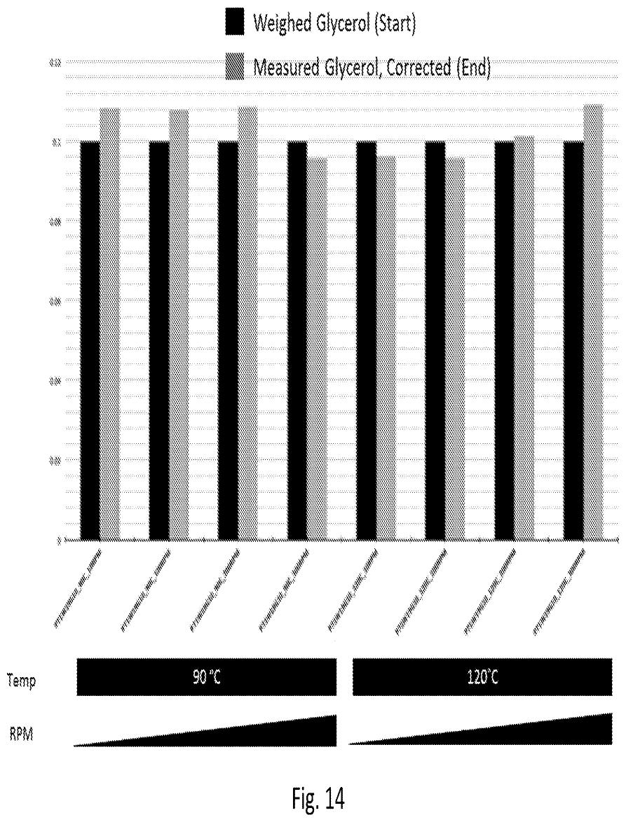

FIG. 14 shows Metabolites data for samples listed above in Table 14 generated under extrusion conditions at 90 and 120° C., where extrudates were obtained for each temperature using operating parameters of 10, 100, 200 and 300 RPM. Glycerol loss was negligible across all treatments.

### Example 8: Metabolites Analysis of Glycerol Content

P49W21G30 and P25W05G70 Silk powder compositions were mixed and subjected to twin screw extrusion as described in Example 2. Extrudates were chopped into pellets and subjected to Melt Flow Indexing (MFI). MFI was conducted on a Goettfert Melt Indexer, Model #MI-40, Serial #10005563. The Barrel diameter was 9.5320 mm, the die length was 8.015 mm with a 2.09 mm orifice diameter. A two minute preheat was utilized. Testing was conducted per ASTM D1238 standard test method, for flow rates of thermoplastics by Extrusion Plastometer. Testing was performed at 95° C. with loads of 2.16 kg or 21.6 kg.

Table 15 shows Melt Flow Index Values obtained from respective material compositions. n=3 for P49W21G30, and n=6 for P25W05G70 tested at 2.1 and 21.1 Kg respectively. ‘+/−’ indicates standard deviation among n samples. The data indicate that Protein/Glycerol/Water based pellets exhibit MFI values that are within a similar range to polypropylene, for example (20 g/10 min). Higher flow rates are obtained at lower protein composition.

TABLE 15
Melt Flow Index Values

| | 2.1 Kg | 21.1 Kg |
|---|---|---|
| P49W21G30 | — | 7.10 +/− 2.58 |
| P25W05G70 | 14.18 +/− 3.07 | — |

1. A composition for a molded body comprising a recombinant spider silk protein and a plasticizer, wherein the composition is capable of being induced into a flowable state, wherein the recombinant spider silk protein is substantially non-degraded in the flowable state.

2. The composition of claim 1, wherein the composition is capable of being induced into the flowable state by the application of shear force and pressure.

3. The composition of claim 2 wherein the composition is capable of being induced into the flowable state by the application of shear force and pressure without the application of heat.

4. The composition of claim 3, wherein the composition is capable of being induced into the flowable state and extruded multiple times with the recombinant spider silk protein remaining substantially non-degraded within the composition.

5. The composition of claim 1 wherein the composition is thermoplastic.

6. The composition of claim 2, wherein the composition is capable of being induced into the flowable state through the application of shear force ranging from 1.5 Nm to 13 Nm.

7. The composition of claim 2, wherein the composition is capable of being induced into the flowable state through the application of shear force ranging from 2 Nm to 6 Nm.

8. The composition of claim 2, wherein the composition is capable of being induced into the flowable state through the application of pressure ranging from 1 MPa to 300 MPa.

9. The composition of claim 2, wherein the composition is capable of being induced into the flowable state through the application of pressure ranging from 5 MPa to 75 MPa.

10. The composition of any one of claims 6-9, wherein said composition is capable of being induced into the flowable state at less than 120° C., less than 80° C., less than 40° C., or at room temperature.

11. The composition of any one of claims 1-10, wherein said composition has a melt flow index of at least 0.5, at least 1, at least 2, or at least 5 as tested per ASTM D1238 at 95° C. with a load of 2.16 kg.

12. The composition of any one of claims 1-10, wherein said composition has a melt flow index of at least 0.5, at least 1, at least 2, or at least 5 as tested per ASTM D1238 at 95° C. with a load of 21.6 kg.

13. The composition of claim 1, wherein the composition is substantially homogeneous.

14. The composition of claim 1, wherein the recombinant spider silk protein comprises repeat units.

15. The composition of claim 1, wherein the recombinant spider silk protein comprises in the range 2 to 20 repeat units of amino acid residue length ranging from 60 to 100 amino acids.

16. The composition of claim 1, wherein the molecular weight of the recombinant spider silk protein ranges from 20 to 2000 kDa.

17. The composition of claim 1 wherein the recombinant spider silk protein comprises at least two occurrences of a repeat unit, the repeat unit comprising:
more than 150 amino acid residues and having a molecular weight of at least 10 kDa;
an alanine-rich region with 6 or more consecutive amino acids, comprising an alanine content of at least 80%; and
a glycine-rich region with 12 or more consecutive amino acids, comprising a glycine content of at least 40% and an alanine content of less than 30%.

18. The composition of claim 1, wherein the plasticizer is selected from a polyol, water and/or urea.

19. The composition of claim 16, wherein the polyol comprises glycerol.

20. The composition of claim 1, wherein the plasticizer comprises water.

21. The composition of claim 1, wherein the recombinant spider silk protein is present in a recombinant spider silk polypeptide powder and wherein the ratio by weight of plasticizer to recombinant silk polypeptide powder ranges from 0.05:1 to 4:1.

22. The composition of claim 1, wherein the recombinant spider silk protein is present in a recombinant spider silk polypeptide powder and the ratio by weight of plasticizer to recombinant silk polypeptide powder ranges from 0.20:1 to 0.70:1.

23. The composition of claim 1, wherein the recombinant spider silk protein is present in a recombinant spider silk polypeptide powder and the amount of recombinant spider silk polypeptide powder in the composition ranges from 1 to 90 wt % recombinant spider silk protein.

24. The composition of claim 1 wherein the recombinant spider silk protein is present in a recombinant spider silk polypeptide powder and the amount of recombinant spider silk polypeptide powder in the composition ranges from 20 to 41 wt % recombinant spider silk protein.

25. The composition of claim 1 comprising in the range 1 to 90 wt % of glycerol as a plasticizer.

26. The composition of claim 1 comprising in the range 15 to 30 wt % of glycerol as a plasticizer.

27. The composition of claim 1 comprising in the range 5 to 80 wt % of water as a plasticizer.

28. The composition of claim 1 comprising in the range 19 to 27 wt % of water as a plasticizer.

29. The composition of claim 1, wherein the recombinant spider silk protein is degraded in an amount of less than 10.0 weight % in the flowable state.

30. The composition of claim 1, wherein the recombinant spider silk protein is degraded in an amount of less than 6.0 weight % in the flowable state.

31. The composition of claim 1, wherein the recombinant spider silk protein is degraded in an amount of less than 2.0 weight % in flowable state.

32. The composition of any of claims 29-31, wherein the degradation of the recombinant spider silk protein is assessed by measuring the amount of full-length recombinant spider silk protein present in the composition before and after the flowable state is induced.

33. The composition of claim 32, wherein the amount of full-length recombinant spider silk protein is measured using size exclusion chromatography.

34. A molded body comprising the composition of any one of claims 1-33.

35. The molded body of claim 34, wherein the molded body is a fiber.

36. The molded body of claim 35, wherein the fiber is of strength in the range of 100 Pa to 1.2 GPa.

37. The molded body of claim 35, wherein the fiber is of birefringence in the range from 5×10⁻⁵ to ˜0.04 as measured by polarized light microscopy.

38. A process for preparing a molded body, comprising the steps of:
(a) applying pressure and shear force to a composition comprising a recombinant spider silk protein and a plasticizer to transform the composition to a flowable state, and
(b) extruding the composition in the flowable state to form a molded body.

39. The process of claim 38, wherein extruding the composition to form a molded body comprises extruding the composition to form a fiber.

40. The process of claims 39, wherein extruding the composition to form a fiber comprises extruding the composition through a spinneret.

41. The process of claim 38, wherein extruding the composition to form a molded body comprises extruding the composition into a mold.

42. The process of claim 38, further comprising:
(a) applying pressure and shear force to the molded body to transform the molded body to a composition in a flowable state, and
(b) extruding the composition in the flowable state to form a second molded body.

43. The process of claim 42, further comprising repeating steps (a) and (b) to the second molded body at least once.

44. The process of any one of claims 38-43, wherein said shear force is from 1.5 to 13 Nm.

45. The process of any one of claims 38-43, wherein said pressure is from 1 MPa to 300 MPa.

46. The process of any one of claims 38-45, wherein the shear force and pressure are applied to the composition using a capillary rheometer or a twin screw extruder.

47. The process of claim 46, wherein the screw speed of the twin screw extruder ranges from 10 to 300 RPM during application of said pressure and shear force.

48. The process of any one of claims 38-45, wherein an instrument used to apply the shear force and pressure comprises a mixing chamber that is coupled to and proximal to an extrusion chamber.

49. The process of claim 48, wherein the composition is heated in the mixing chamber.

50. The process of claim 48, wherein the composition is heated in the extrusion chamber.

51. The process of either claim 49 or claim 50, wherein the composition is heated to a temperature of less than 120° C.

52. The process of claim 49 or claim 50, wherein the composition is heated to a temperature of less than 80° C.

53. The process of claim 49 or claim 50, wherein the composition is heated to a temperature of less than 40° C.

54. The process of any one of claims 38-53, wherein the molded body after extrusion has a loss of water content of less than 15% as compared to the composition before extrusion.

55. The process of any one of claims 38-53, wherein the molded body after extrusion has a loss of water content of less than 10% as compared to the composition before extrusion.

56. The process of claim 48, wherein the composition has a residence time in the mixing chamber ranging from 3 to 7 minutes.

57. The process of claim 48, wherein the extrusion chamber is tapered proximal to an orifice through which the composition is extruded.

58. The process of claim 48, wherein the extrusion chamber is temperature controlled.

59. The process of any one of claims 48-58, wherein the molded body is a fiber and the fiber is hand drawn.

60. The process of any one of claims 48-59, wherein the molded body is a fiber and the fiber is drawn over multiple steps.

61. The process of any one of claims 48-60, wherein the recombinant spider silk protein is substantially non-degraded in the molded body.

62. The process of claim 61, wherein the recombinant spider silk protein is degraded in amount of less than 10% by weight in the molded body.

63. The process of claim 61, wherein the recombinant spider silk protein is degraded in amount of less than 6% by weight in the molded body.

64. The process of claim 61, wherein the recombinant spider silk protein is degraded in amount of less than 2% by weight in the molded body.

65. The process of any of claims 61-64, wherein the degradation of the recombinant spider silk protein is assessed by measuring the amount of full-length recombinant spider silk protein present in the composition before and after extrusion.

66. The process of claim 65, wherein the amount of full-length recombinant spider silk protein is measured using size exclusion chromatography.

67. The process of any one of claims 38-66, wherein the molded body has minimal birefringence as measured by polarized light microscopy.


SEQUENCE LISTING (38 sequences)

SEQ ID NO: 1 - protein, 945 aa, synthetic construct
  REGION 1..945: note = Description of Artificial Sequence: Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 315 aa, synthetic construct
  REGION 1..315: note = Description of Artificial Sequence: Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 3 - protein, 5 aa, unidentified
  REGION 1..5: note = Description of Unknown: Silk polypeptide block sequence
SGAGG

SEQ ID NO: 4 - protein, 5 aa, unidentified
  REGION 1..5: note = Description of Unknown: Silk polypeptide block sequence
GSGAG

SEQ ID NO: 5 - protein, 5 aa, unidentified
  REGION 1..5: note = Description of Unknown: Silk polypeptide block sequence
GGSGA

SEQ ID NO: 6 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 7 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 8 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 9 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 10 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 11 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 12 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 13 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 14 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 15 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 16 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 17 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 18 - protein, 32 aa, Euprosthenops australis
GGQGGQGQGRYGQGAGSSAAAAAAAAAAAAAA

SEQ ID NO: 19 - protein, 42 aa, Tetragnatha kauaiensis
GGLGGGQGAGQGGQQGAGQGGYGSGLGGAGQGASAAAAAAAA

SEQ ID NO: 20 - protein, 42 aa, Argiope aurantia
GGYGPGAGQQGPGSQGPGSGGQQGPGGLGPYGPSAAAAAAAA

SEQ ID NO: 21 - protein, 46 aa, Deinopis spinosa
GPGGYGGPGQQGPGQGQYGPGTGQQGQGPSGQQGPAGAAAAAAAAA

SEQ ID NO: 22 - protein, 42 aa, Nephila clavata
GPGGYGLGQQGPGQQGPGQQGPAGYGPSGLSGPGGAAAAAAA

SEQ ID NO: 23 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 24 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 25 - protein, 161 aa, Nephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 26 - protein, 186 aa, Nephilengys cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 27 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 28 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 29 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 30 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 31 - protein, 177 aa, Nephilengys cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 32 - protein, 174 aa, Nephilengys cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 33 - protein, 8 aa, synthetic construct
  REGION 1..8: note = Description of Artificial Sequence: Synthetic His tag
  REGION 1..8: note = MISC_FEATURE - This sequence may encompass 6-8 residues
HHHHHHHH

SEQ ID NO: 34 - protein, 1600 aa, synthetic construct
  REGION 1..1600: note = Description of Artificial Sequence: Synthetic polypeptide
  REGION 7..11: note = MISC_FEATURE - This region may encompass "SGGQQ," "GAGQQ," "GQGPY," "AGQQ" or "SQ," wherein some positions may be absent
  REGION 15..19: note = MISC_FEATURE - This region may encompass "SGGQQ," "GAGQQ," "GQGPY," "AGQQ" or "SQ," wherein some positions may be absent
  REGION 23..27: note = MISC_FEATURE - This region may encompass "SGGQQ," "GAGQQ," "GQGPY," "AGQQ" or "SQ," wherein some positions may be absent
  REGION 31..35: note = MISC_FEATURE - This region may encompass "SGGQQ," "GAGQQ," "GQGPY," "AGQQ" or "SQ," wherein some positions may be absent
  REGION 39..43: note = MISC_FEATURE - This region may encompass "SGGQQ," "GAGQQ," "GQGPY," "AGQQ" or "SQ," wherein some positions may be absent
  REGION 47..51: note = MISC_FEATURE - This region may encompass "SGGQQ," "GAGQQ," "GQGPY," "AGQQ" or "SQ," wherein some positions may be absent
  REGION 55..59: note = MISC_FEATURE - This region may encompass "SGGQQ," "GAGQQ," "GQGPY," "AGQQ" or "SQ," wherein some positions may be absent
  REGION 63..67: note = MISC_FEATURE - This region may encompass "SGGQQ," "GAGQQ," "GQGPY," "AGQQ" or "SQ," wherein some positions may be absent
  REGION 4..67: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 71..80: note = MISC_FEATURE - This region may encompass 6-10 residues
  REGION 87..91: note = MISC_FEATURE - This region may encompass "SGGQQ," "GAGQQ," "GQGPY," "AGQQ" or "SQ," wherein some positions may be absent
  REGION 95..99: note = MISC_FEATURE - This region may encompass "SGGQQ," "GAGQQ," "GQGPY," "AGQQ" or "SQ," wherein some positions may be absent
  REGION 103..107: note = MISC_FEATURE - This region may encompass "SGGQQ," "GAGQQ," "GQGPY," "AGQQ" or "SQ," wherein some positions may be absent
  REGION 111..115: note = MISC_FEATURE - This region may encompass "SGGQQ," "GAGQQ," "GQGPY," "AGQQ" or "SQ," wherein some positions may be absent
  REGION 119..123: note = MISC_FEATURE - This region may encompass "SGGQQ," "GAGQQ," "GQGPY," "AGQQ" or "SQ," wherein some positions may be absent
  REGION 127..131: note = MISC_FEATURE - This region may encompass "SGGQQ," "GAGQQ," "GQGPY," "AGQQ" or "SQ," wherein some positions may be absent
  REGION 135..139: note = MISC_FEATURE - This region may encompass "SGGQQ," "GAGQQ," "GQGPY," "AGQQ" or "SQ," wherein some positions may be absent
  REGION 143..147: note = MISC_FEATURE - This region may encompass "SGGQQ," "GAGQQ," "GQGPY," "AGQQ" or "SQ," wherein some positions may be absent
  REGION 84..147: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 151..160: note = MISC_FEATURE - This region may encompass 6-10 residues
  REGION 167..171: note = MISC_FEATURE - This region may encompass "SGGQQ," "GAGQQ," "GQGPY," "AGQQ" or "SQ," wherein some positions may be absent
  REGION 175..179: note = MISC_FEATURE - This region may encompass "SGGQQ," "GAGQQ," "GQGPY," "AGQQ" or "SQ," wherein some positions may be absent
  REGION 183..187: note = MISC_FEATURE - This region may encompass "SGGQQ," "GAGQQ," "GQGPY," "AGQQ" or "SQ," wherein some positions may be absent
  REGION 191..195: note = MISC_FEATURE - This region may encompass "SGGQQ," "GAGQQ," "GQGPY," "AGQQ" or "SQ," wherein some positions may be absent
  REGION 199..203: note = MISC_FEATURE - This region may encompass "SGGQQ," "GAGQQ," "GQGPY," "AGQQ" or "SQ," wherein some positions may be absent
  REGION 207..211: note = MISC_FEATURE - This region may encompass "SGGQQ," "GAGQQ," "GQGPY," "AGQQ" or "SQ," wherein some positions may be absent
  REGION 215..219: note = MISC_FEATURE - This region may encompass "SGGQQ," "GAGQQ," "GQGPY," "AGQQ" or "SQ," wherein some positions may be absent
  REGION 223..227: note = MISC_FEATURE - This region may encompass "SGGQQ," "GAGQQ," "GQGPY," "AGQQ" or "SQ," wherein some positions may be absent
  REGION 164..227: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 231..240: note = MISC_FEATURE - This region may encompass 6-10 residues
  REGION 247..251: note = MISC_FEATURE - This region may encompass "SGGQQ," "GAGQQ," "GQGPY," "AGQQ" or "SQ," wherein some positions may be absent
  REGION 255..259: note = MISC_FEATURE - This region may encompass "SGGQQ," "GAGQQ," "GQGPY," "AGQQ" or "SQ," wherein some positions may be absent
  REGION 263..267: note = MISC_FEATURE - This region may encompass "SGGQQ," "GAGQQ," "GQGPY," "AGQQ" or "SQ," wherein some positions may be absent
  REGION 271..275: note = MISC_FEATURE - This region may encompass "SGGQQ," "GAGQQ," "GQGPY," "AGQQ" or "SQ," wherein some positions may be absent
  REGION 279..283: note = MISC_FEATURE - This region may encompass "SGGQQ," "GAGQQ," "GQGPY," "AGQQ" or "SQ," wherein some positions may be absent
  REGION 287..291: note = MISC_FEATURE - This region may encompass "SGGQQ," "GAGQQ," "GQGPY," "AGQQ" or "SQ," wherein some positions may be absent
  REGION 295..299: note = MISC_FEATURE - This region may encompass "SGGQQ," "GAGQQ," "GQGPY," "AGQQ" or "SQ," wherein some positions may be absent
  REGION 303..307: note = MISC_FEATURE - This region may encompass "SGGQQ," "GAGQQ," "GQGPY," "AGQQ" or "SQ," wherein some positions may be absent
  REGION 244..307: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 311..320: note = MISC_FEATURE - This region may encompass 6-10 residues
  REGION 327..331: note = MISC_FEATURE - This region may encompass "SGGQQ," "GAGQQ," "GQGPY," "AGQQ" or "SQ," wherein some positions may be absent
  REGION 335..339: note = MISC_FEATURE - This region may encompass "SGGQQ," "GAGQQ," "GQGPY," "AGQQ" or "SQ," wherein some positions may be absent
  REGION 343..347: note = MISC_FEATURE - This region may encompass "SGGQQ," "GAGQQ," "GQGPY," "AGQQ" or "SQ," wherein some positions may be absent
  REGION 351..355: note = MISC_FEATURE - This region may encompass "SGGQQ," "GAGQQ," "GQGPY," "AGQQ" or "SQ," wherein some positions may be absent
  REGION 359..363: note = MISC_FEATURE - This region may encompass "SGGQQ," "GAGQQ," "GQGPY," "AGQQ" or "SQ," wherein some positions may be absent
  REGION 367..371: note = MISC_FEATURE - This region may encompass "SGGQQ," "GAGQQ," "GQGPY," "AGQQ" or "SQ," wherein some positions may be absent
  REGION 375..379: note = MISC_FEATURE - This region may encompass "SGGQQ," "GAGQQ," "GQGPY," "AGQQ" or "SQ," wherein some positions may be absent
  REGION 383..387: note = MISC_FEATURE - This region may encompass "SGGQQ," "GAGQQ," "GQGPY," "AGQQ" or "SQ," wherein some positions may be absent
  REGION 324..387: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 391..400: note = MISC_FEATURE - This region may encompass 6-10 residues
  REGION 407..411: note = MISC_FEATURE - This region may encompass "SGGQQ," "GAGQQ," "GQGPY," "AGQQ" or "SQ," wherein some positions may be absent
  REGION 415..419: note = MISC_FEATURE - This region may encompass "SGGQQ," "GAGQQ," "GQGPY," "AGQQ" or "SQ," wherein some positions may be absent
  REGION 423..427: note = MISC_FEATURE - This region may encompass "SGGQQ," "GAGQQ," "GQGPY," "AGQQ" or "SQ," wherein some positions may be absent
  REGION 431..435: note = MISC_FEATURE - This region may encompass "SGGQQ," "GAGQQ," "GQGPY," "AGQQ" or "SQ," wherein some positions may be absent
  REGION 439..443: note = MISC_FEATURE - This region may encompass "SGGQQ," "GAGQQ," "GQGPY," "AGQQ" or "SQ," wherein some positions may be absent
  REGION 447..451: note = MISC_FEATURE - This region may encompass "SGGQQ," "GAGQQ," "GQGPY," "AGQQ" or "SQ," wherein some positions may be absent
  REGION 455..459: note = MISC_FEATURE - This region may encompass "SGGQQ," "GAGQQ," "GQGPY," "AGQQ" or "SQ," wherein some positions may be absent
  REGION 463..467: note = MISC_FEATURE - This region may encompass "SGGQQ," "GAGQQ," "GQGPY," "AGQQ" or "SQ," wherein some positions may be absent
  REGION 404..467: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 471..480: note = MISC_FEATURE - This region may encompass 6-10 residues
  REGION 487..491: note = MISC_FEATURE - This region may encompass "SGGQQ," "GAGQQ," "GQGPY," "AGQQ" or "SQ," wherein some positions may be absent
  REGION 495..499: note = MISC_FEATURE - This region may encompass "SGGQQ," "GAGQQ," "GQGPY," "AGQQ" or "SQ," wherein some positions may be absent
  REGION 503..507: note = MISC_FEATURE - This region may encompass "SGGQQ," "GAGQQ," "GQGPY," "AGQQ" or "SQ," wherein some positions may be absent
  REGION 511..515: note = MISC_FEATURE - This region may encompass "SGGQQ," "GAGQQ," "GQGPY," "AGQQ" or "SQ," wherein some positions may be absent
  REGION 519..523: note = MISC_FEATURE - This region may encompass "SGGQQ," "GAGQQ," "GQGPY," "AGQQ" or "SQ," wherein some positions may be absent
  REGION 527..531: note = MISC_FEATURE - This region may encompass "SGGQQ," "GAGQQ," "GQGPY," "AGQQ" or "SQ," wherein some positions may be absent
  REGION 535..539: note = MISC_FEATURE - This region may encompass "SGGQQ," "GAGQQ," "GQGPY," "AGQQ" or "SQ," wherein some positions may be absent
  REGION 543..547: note = MISC_FEATURE - This region may encompass "SGGQQ," "GAGQQ," "GQGPY," "AGQQ" or "SQ," wherein some positions may be absent
  REGION 484..547: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 551..560: note = MISC_FEATURE - This region may encompass 6-10 residues
  REGION 567..571: note = MISC_FEATURE - This region may encompass "SGGQQ," "GAGQQ," "GQGPY," "AGQQ" or "SQ," wherein some positions may be absent
  REGION 575..579: note = MISC_FEATURE - This region may encompass "SGGQQ," "GAGQQ," "GQGPY," "AGQQ" or "SQ," wherein some positions may be absent
  REGION 583..587: note = MISC_FEATURE - This region may encompass "SGGQQ," "GAGQQ," "GQGPY," "AGQQ" or "SQ," wherein some positions may be absent
  REGION 591..595: note = MISC_FEATURE - This region may encompass "SGGQQ," "GAGQQ," "GQGPY," "AGQQ" or "SQ," wherein some positions may be absent
  REGION 599..603: note = MISC_FEATURE - This region may encompass "SGGQQ," "GAGQQ," "GQGPY," "AGQQ" or "SQ," wherein some positions may be absent
  REGION 607..611: note = MISC_FEATURE - This region may encompass "SGGQQ," "GAGQQ," "GQGPY," "AGQQ" or "SQ," wherein some positions may be absent
  REGION 615..619: note = MISC_FEATURE - This region may encompass "SGGQQ," "GAGQQ," "GQGPY," "AGQQ" or "SQ," wherein some positions may be absent
  REGION 623..627: note = MISC_FEATURE - This region may encompass "SGGQQ," "GAGQQ," "GQGPY," "AGQQ" or "SQ," wherein some positions may be absent
  REGION 564..627: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 631..640: note = MISC_FEATURE - This region may encompass 6-10 residues
  REGION 647..651: note = MISC_FEATURE - This region may encompass "SGGQQ," "GAGQQ," "GQGPY," "AGQQ" or "SQ," wherein some positions may be absent
  REGION 655..659: note = MISC_FEATURE - This region may encompass "SGGQQ," "GAGQQ," "GQGPY," "AGQQ" or "SQ," wherein some positions may be absent
  REGION 663..667: note = MISC_FEATURE - This region may encompass "SGGQQ," "GAGQQ," "GQGPY," "AGQQ" or "SQ," wherein some positions may be absent
  REGION 671..675: note = MISC_FEATURE - This region may encompass "SGGQQ," "GAGQQ," "GQGPY," "AGQQ" or "SQ," wherein some positions may be absent
  REGION 679..683: note = MISC_FEATURE - This region may encompass "SGGQQ," "GAGQQ," "GQGPY," "AGQQ" or "SQ," wherein some positions may be absent
  REGION 687..691: note = MISC_FEATURE - This region may encompass "SGGQQ," "GAGQQ," "GQGPY," "AGQQ" or "SQ," wherein some positions may be absent
  REGION 695..699: note = MISC_FEATURE - This region may encompass "SGGQQ," "GAGQQ," "GQGPY," "AGQQ" or "SQ," wherein some positions may be absent
  REGION 703..707: note = MISC_FEATURE - This region may encompass "SGGQQ," "GAGQQ," "GQGPY," "AGQQ" or "SQ," wherein some positions may be absent
  REGION 644..707: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 711..720: note = MISC_FEATURE - This region may encompass 6-10 residues
  REGION 727..731: note = MISC_FEATURE - This region may encompass "SGGQQ," "GAGQQ," "GQGPY," "AGQQ" or "SQ," wherein some positions may be absent
  REGION 735..739: note = MISC_FEATURE - This region may encompass "SGGQQ," "GAGQQ," "GQGPY," "AGQQ" or "SQ," wherein some positions may be absent
  REGION 743..747: note = MISC_FEATURE - This region may encompass "SGGQQ," "GAGQQ," "GQGPY," "AGQQ" or "SQ," wherein some positions may be absent
  REGION 751..755: note = MISC_FEATURE - This region may encompass "SGGQQ," "GAGQQ," "GQGPY," "AGQQ" or "SQ," wherein some positions may be absent
  REGION 759..763: note = MISC_FEATURE - This region may encompass "SGGQQ," "GAGQQ," "GQGPY," "AGQQ" or "SQ," wherein some positions may be absent
  REGION 767..771: note = MISC_FEATURE - This region may encompass "SGGQQ," "GAGQQ," "GQGPY," "AGQQ" or "SQ," wherein some positions may be absent
  REGION 775..779: note = MISC_FEATURE - This region may encompass "SGGQQ," "GAGQQ," "GQGPY," "AGQQ" or "SQ," wherein some positions may be absent
  REGION 783..787: note = MISC_FEATURE - This region may encompass "SGGQQ," "GAGQQ," "GQGPY," "AGQQ" or "SQ," wherein some positions may be absent
  REGION 724..787: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 791..800: note = MISC_FEATURE - This region may encompass 6-10 residues
  REGION 807..811: note = MISC_FEATURE - This region may encompass "SGGQQ," "GAGQQ," "GQGPY," "AGQQ" or "SQ," wherein some positions may be absent
  REGION 815..819: note = MISC_FEATURE - This region may encompass "SGGQQ," "GAGQQ," "GQGPY," "AGQQ" or "SQ," wherein some positions may be absent
  REGION 823..827: note = MISC_FEATURE - This region may encompass "SGGQQ," "GAGQQ," "GQGPY," "AGQQ" or "SQ," wherein some positions may be absent
  REGION 831..835: note = MISC_FEATURE - This region may encompass "SGGQQ," "GAGQQ," "GQGPY," "AGQQ" or "SQ," wherein some positions may be absent
  REGION 839..843: note = MISC_FEATURE - This region may encompass "SGGQQ," "GAGQQ," "GQGPY," "AGQQ" or "SQ," wherein some positions may be absent
  REGION 847..851: note = MISC_FEATURE - This region may encompass "SGGQQ," "GAGQQ," "GQGPY," "AGQQ" or "SQ," wherein some positions may be absent
  REGION 855..859: note = MISC_FEATURE - This region may encompass "SGGQQ," "GAGQQ," "GQGPY," "AGQQ" or "SQ," wherein some positions may be absent
  REGION 863..867: note = MISC_FEATURE - This region may encompass "SGGQQ," "GAGQQ," "GQGPY," "AGQQ" or "SQ," wherein some positions may be absent
  REGION 804..867: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 871..880: note = MISC_FEATURE - This region may encompass 6-10 residues
  REGION 887..891: note = MISC_FEATURE - This region may encompass "SGGQQ," "GAGQQ," "GQGPY," "AGQQ" or "SQ," wherein some positions may be absent
  REGION 895..899: note = MISC_FEATURE - This region may encompass "SGGQQ," "GAGQQ," "GQGPY," "AGQQ" or "SQ," wherein some positions may be absent
  REGION 903..907: note = MISC_FEATURE - This region may encompass "SGGQQ," "GAGQQ," "GQGPY," "AGQQ" or "SQ," wherein some positions may be absent
  REGION 911..915: note = MISC_FEATURE - This region may encompass "SGGQQ," "GAGQQ," "GQGPY," "AGQQ" or "SQ," wherein some positions may be absent
  REGION 919..923: note = MISC_FEATURE - This region may encompass "SGGQQ," "GAGQQ," "GQGPY," "AGQQ" or "SQ," wherein some positions may be absent
  REGION 927..931: note = MISC_FEATURE - This region may encompass "SGGQQ," "GAGQQ," "GQGPY," "AGQQ" or "SQ," wherein some positions may be absent
  REGION 935..939: note = MISC_FEATURE - This region may encompass "SGGQQ," "GAGQQ," "GQGPY," "AGQQ" or "SQ," wherein some positions may be absent
  REGION 943..947: note = MISC_FEATURE - This region may encompass "SGGQQ," "GAGQQ," "GQGPY," "AGQQ" or "SQ," wherein some positions may be absent
  REGION 884..947: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 951..960: note = MISC_FEATURE - This region may encompass 6-10 residues
  REGION 967..971: note = MISC_FEATURE - This region may encompass "SGGQQ," "GAGQQ," "GQGPY," "AGQQ" or "SQ," wherein some positions may be absent
  REGION 975..979: note = MISC_FEATURE - This region may encompass "SGGQQ," "GAGQQ," "GQGPY," "AGQQ" or "SQ," wherein some positions may be absent
  REGION 983..987: note = MISC_FEATURE - This region may encompass "SGGQQ," "GAGQQ," "GQGPY," "AGQQ" or "SQ," wherein some positions may be absent
  REGION 991..995: note = MISC_FEATURE - This region may encompass "SGGQQ," "GAGQQ," "GQGPY," "AGQQ" or "SQ," wherein some positions may be absent
  REGION 999..1003: note = MISC_FEATURE - This region may encompass "SGGQQ," "GAGQQ," "GQGPY," "AGQQ" or "SQ," wherein some positions may be absent
  REGION 1007..1011: note = MISC_FEATURE - This region may encompass "SGGQQ," "GAGQQ," "GQGPY," "AGQQ" or "SQ," wherein some positions may be absent
  REGION 1015..1019: note = MISC_FEATURE - This region may encompass "SGGQQ," "GAGQQ," "GQGPY," "AGQQ" or "SQ," wherein some positions may be absent
  REGION 1023..1027: note = MISC_FEATURE - This region may encompass "SGGQQ," "GAGQQ," "GQGPY," "AGQQ" or "SQ," wherein some positions may be absent
  REGION 964..1027: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1031..1040: note = MISC_FEATURE - This region may encompass 6-10 residues
  REGION 1047..1051: note = MISC_FEATURE - This region may encompass "SGGQQ," "GAGQQ," "GQGPY," "AGQQ" or "SQ," wherein some positions may be absent
  REGION 1055..1059: note = MISC_FEATURE - This region may encompass "SGGQQ," "GAGQQ," "GQGPY," "AGQQ" or "SQ," wherein some positions may be absent
  REGION 1063..1067: note = MISC_FEATURE - This region may encompass "SGGQQ," "GAGQQ," "GQGPY," "AGQQ" or "SQ," wherein some positions may be absent
  REGION 1071..1075: note = MISC_FEATURE - This region may encompass "SGGQQ," "GAGQQ," "GQGPY," "AGQQ" or "SQ," wherein some positions may be absent
  REGION 1079..1083: note = MISC_FEATURE - This region may encompass "SGGQQ," "GAGQQ," "GQGPY," "AGQQ" or "SQ," wherein some positions may be absent
  REGION 1087..1091: note = MISC_FEATURE - This region may encompass "SGGQQ," "GAGQQ," "GQGPY," "AGQQ" or "SQ," wherein some positions may be absent
  REGION 1095..1099: note = MISC_FEATURE - This region may encompass "SGGQQ," "GAGQQ," "GQGPY," "AGQQ" or "SQ," wherein some positions may be absent
  REGION 1103..1107: note = MISC_FEATURE - This region may encompass "SGGQQ," "GAGQQ," "GQGPY," "AGQQ" or "SQ," wherein some positions may be absent
  REGION 1044..1107: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1111..1120: note = MISC_FEATURE - This region may encompass 6-10 residues
  REGION 1127..1131: note = MISC_FEATURE - This region may encompass "SGGQQ," "GAGQQ," "GQGPY," "AGQQ" or "SQ," wherein some positions may be absent
  REGION 1135..1139: note = MISC_FEATURE - This region may encompass "SGGQQ," "GAGQQ," "GQGPY," "AGQQ" or "SQ," wherein some positions may be absent
  REGION 1143..1147: note = MISC_FEATURE - This region may encompass "SGGQQ," "GAGQQ," "GQGPY," "AGQQ" or "SQ," wherein some positions may be absent
  REGION 1151..1155: note = MISC_FEATURE - This region may encompass "SGGQQ," "GAGQQ," "GQGPY," "AGQQ" or "SQ," wherein some positions may be absent
  REGION 1159..1163: note = MISC_FEATURE - This region may encompass "SGGQQ," "GAGQQ," "GQGPY," "AGQQ" or "SQ," wherein some positions may be absent
  REGION 1167..1171: note = MISC_FEATURE - This region may encompass "SGGQQ," "GAGQQ," "GQGPY," "AGQQ" or "SQ," wherein some positions may be absent
  REGION 1175..1179: note = MISC_FEATURE - This region may encompass "SGGQQ," "GAGQQ," "GQGPY," "AGQQ" or "SQ," wherein some positions may be absent
  REGION 1183..1187: note = MISC_FEATURE - This region may encompass "SGGQQ," "GAGQQ," "GQGPY," "AGQQ" or "SQ," wherein some positions may be absent
  REGION 1124..1187: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1191..1200: note = MISC_FEATURE - This region may encompass 6-10 residues
  REGION 1207..1211: note = MISC_FEATURE - This region may encompass "SGGQQ," "GAGQQ," "GQGPY," "AGQQ" or "SQ," wherein some positions may be absent
  REGION 1215..1219: note = MISC_FEATURE - This region may encompass "SGGQQ," "GAGQQ," "GQGPY," "AGQQ" or "SQ," wherein some positions may be absent
  REGION 1223..1227: note = MISC_FEATURE - This region may encompass "SGGQQ," "GAGQQ," "GQGPY," "AGQQ" or "SQ," wherein some positions may be absent
  REGION 1231..1235: note = MISC_FEATURE - This region may encompass "SGGQQ," "GAGQQ," "GQGPY," "AGQQ" or "SQ," wherein some positions may be absent
  REGION 1239..1243: note = MISC_FEATURE - This region may encompass "SGGQQ," "GAGQQ," "GQGPY," "AGQQ" or "SQ," wherein some positions may be absent
  REGION 1247..1251: note = MISC_FEATURE - This region may encompass "SGGQQ," "GAGQQ," "GQGPY," "AGQQ" or "SQ," wherein some positions may be absent
  REGION 1255..1259: note = MISC_FEATURE - This region may encompass "SGGQQ," "GAGQQ," "GQGPY," "AGQQ" or "SQ," wherein some positions may be absent
  REGION 1263..1267: note = MISC_FEATURE - This region may encompass "SGGQQ," "GAGQQ," "GQGPY," "AGQQ" or "SQ," wherein some positions may be absent
  REGION 1204..1267: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1271..1280: note = MISC_FEATURE - This region may encompass 6-10 residues
  REGION 1287..1291: note = MISC_FEATURE - This region may encompass "SGGQQ," "GAGQQ," "GQGPY," "AGQQ" or "SQ," wherein some positions may be absent
  REGION 1295..1299: note = MISC_FEATURE - This region may encompass "SGGQQ," "GAGQQ," "GQGPY," "AGQQ" or "SQ," wherein some positions may be absent
  REGION 1303..1307: note = MISC_FEATURE - This region may encompass "SGGQQ," "GAGQQ," "GQGPY," "AGQQ" or "SQ," wherein some positions may be absent
  REGION 1311..1315: note = MISC_FEATURE - This region may encompass "SGGQQ," "GAGQQ," "GQGPY," "AGQQ" or "SQ," wherein some positions may be absent
  REGION 1319..1323: note = MISC_FEATURE - This region may encompass "SGGQQ," "GAGQQ," "GQGPY," "AGQQ" or "SQ," wherein some positions may be absent
  REGION 1327..1331: note = MISC_FEATURE - This region may encompass "SGGQQ," "GAGQQ," "GQGPY," "AGQQ" or "SQ," wherein some positions may be absent
  REGION 1335..1339: note = MISC_FEATURE - This region may encompass "SGGQQ," "GAGQQ," "GQGPY," "AGQQ" or "SQ," wherein some positions may be absent
  REGION 1343..1347: note = MISC_FEATURE - This region may encompass "SGGQQ," "GAGQQ," "GQGPY," "AGQQ" or "SQ," wherein some positions may be absent
  REGION 1284..1347: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1351..1360: note = MISC_FEATURE - This region may encompass 6-10 residues
  REGION 1367..1371: note = MISC_FEATURE - This region may encompass "SGGQQ," "GAGQQ," "GQGPY," "AGQQ" or "SQ," wherein some positions may be absent
  REGION 1375..1379: note = MISC_FEATURE - This region may encompass "SGGQQ," "GAGQQ," "GQGPY," "AGQQ" or "SQ," wherein some positions may be absent
  REGION 1383..1387: note = MISC_FEATURE - This region may encompass "SGGQQ," "GAGQQ," "GQGPY," "AGQQ" or "SQ," wherein some positions may be absent
  REGION 1391..1395: note = MISC_FEATURE - This region may encompass "SGGQQ," "GAGQQ," "GQGPY," "AGQQ" or "SQ," wherein some positions may be absent
  REGION 1399..1403: note = MISC_FEATURE - This region may encompass "SGGQQ," "GAGQQ," "GQGPY," "AGQQ" or "SQ," wherein some positions may be absent
  REGION 1407..1411: note = MISC_FEATURE - This region may encompass "SGGQQ," "GAGQQ," "GQGPY," "AGQQ" or "SQ," wherein some positions may be absent
  REGION 1415..1419: note = MISC_FEATURE - This region may encompass "SGGQQ," "GAGQQ," "GQGPY," "AGQQ" or "SQ," wherein some positions may be absent
  REGION 1423..1427: note = MISC_FEATURE - This region may encompass "SGGQQ," "GAGQQ," "GQGPY," "AGQQ" or "SQ," wherein some positions may be absent
  REGION 1364..1427: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1431..1440: note = MISC_FEATURE - This region may encompass 6-10 residues
  REGION 1447..1451: note = MISC_FEATURE - This region may encompass "SGGQQ," "GAGQQ," "GQGPY," "AGQQ" or "SQ," wherein some positions may be absent
  REGION 1455..1459: note = MISC_FEATURE - This region may encompass "SGGQQ," "GAGQQ," "GQGPY," "AGQQ" or "SQ," wherein some positions may be absent
  REGION 1463..1467: note = MISC_FEATURE - This region may encompass "SGGQQ," "GAGQQ," "GQGPY," "AGQQ" or "SQ," wherein some positions may be absent
  REGION 1471..1475: note = MISC_FEATURE - This region may encompass "SGGQQ," "GAGQQ," "GQGPY," "AGQQ" or "SQ," wherein some positions may be absent
  REGION 1479..1483: note = MISC_FEATURE - This region may encompass "SGGQQ," "GAGQQ," "GQGPY," "AGQQ" or "SQ," wherein some positions may be absent
  REGION 1487..1491: note = MISC_FEATURE - This region may encompass "SGGQQ," "GAGQQ," "GQGPY," "AGQQ" or "SQ," wherein some positions may be absent
  REGION 1495..1499: note = MISC_FEATURE - This region may encompass "SGGQQ," "GAGQQ," "GQGPY," "AGQQ" or "SQ," wherein some positions may be absent
  REGION 1503..1507: note = MISC_FEATURE - This region may encompass "SGGQQ," "GAGQQ," "GQGPY," "AGQQ" or "SQ," wherein some positions may be absent
  REGION 1444..1507: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1511..1520: note = MISC_FEATURE - This region may encompass 6-10 residues
  REGION 1527..1531: note = MISC_FEATURE - This region may encompass "SGGQQ," "GAGQQ," "GQGPY," "AGQQ" or "SQ," wherein some positions may be absent
  REGION 1535..1539: note = MISC_FEATURE - This region may encompass "SGGQQ," "GAGQQ," "GQGPY," "AGQQ" or "SQ," wherein some positions may be absent
  REGION 1543..1547: note = MISC_FEATURE - This region may encompass "SGGQQ," "GAGQQ," "GQGPY," "AGQQ" or "SQ," wherein some positions may be absent
  REGION 1551..1555: note = MISC_FEATURE - This region may encompass "SGGQQ," "GAGQQ," "GQGPY," "AGQQ" or "SQ," wherein some positions may be absent
  REGION 1559..1563: note = MISC_FEATURE - This region may encompass "SGGQQ," "GAGQQ," "GQGPY," "AGQQ" or "SQ," wherein some positions may be absent
  REGION 1567..1571: note = MISC_FEATURE - This region may encompass "SGGQQ," "GAGQQ," "GQGPY," "AGQQ" or "SQ," wherein some positions may be absent
  REGION 1575..1579: note = MISC_FEATURE - This region may encompass "SGGQQ," "GAGQQ," "GQGPY," "AGQQ" or "SQ," wherein some positions may be absent
  REGION 1583..1587: note = MISC_FEATURE - This region may encompass "SGGQQ," "GAGQQ," "GQGPY," "AGQQ" or "SQ," wherein some positions may be absent
  REGION 1524..1587: note = MISC_FEATURE - This region may encompass 4-8 repeating "GPG-X1" repeating units, wherein X1 is "SGGQQ," "GAGQQ," "GQGPY," "AGQQ" or "SQ," and some positions may be absent
  REGION 1591..1600: note = MISC_FEATURE - This region may encompass 6-10 residues
  REGION 1..1600: note = MISC_FEATURE - This sequence may encompass 2-20 "GGY-[GPG-X1]n1-GPS-(A)n2" repeating units, wherein X1 is "SGGQQ," "GAGQQ," "GQGPY," "AGQQ" or "SQ," n1 is 4-8 and n2 is 6-10 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAA

SEQ ID NO: 35 - protein, 5 aa, synthetic construct
  REGION 1..5: note = Description of Artificial Sequence: Synthetic peptide
SGGQQ

SEQ ID NO: 36 - protein, 5 aa, synthetic construct
  REGION 1..5: note = Description of Artificial Sequence: Synthetic peptide
GAGQQ

SEQ ID NO: 37 - protein, 5 aa, synthetic construct
  REGION 1..5: note = Description of Artificial Sequence: Synthetic peptide
GQGPY

SEQ ID NO: 38 - protein, 4 aa, synthetic construct
  REGION 1..4: note = Description of Artificial Sequence: Synthetic peptide
AGQQ